(19) United States
(12) Patent Application Publication
(10) Pub. No.: US 2023/0414369 A1
(43) Pub. Date: Dec. 28, 2023

(54) AN ORTHOPAEDIC IMPLANT AND A SURGICAL ORTHOPAEDIC SYSTEM INCORPORATING SAME

(71) Applicant: AUSTRALIAN INSTITUTE OF ROBOTIC ORTHOPAEDICS PTY LTD, Nedlands (AU)

(72) Inventors: Riaz Jan Kjell Khan, Cottesloe (AU); Daniel Paul Fick, Cottesloe (AU); Brett Robertson, Fremantle (AU); Richard Tony Chipper, Mount Claremont (AU)

(21) Appl. No.: 17/921,527

(22) PCT Filed: Apr. 28, 2021
(86) PCT No.: PCT/AU2021/050386

§ 371 Date: Oct. 26, 2022

(30) Foreign Application Priority Data
Apr. 30, 2020 (AU) 2020901373

(51) Int. Cl. A61F 2/30; A61F 2/38; A61B 18/20

(52) U.S. Cl. CPC A61F 2/30771; A61F 2/3859; A61F 2/3877; A61F 2/389; A61B 18/20; A61B 2018/00577

(57) ABSTRACT

An orthopaedic implant for use with or as part of an orthopaedic implant system, comprising: a body having a bone facing surface configured to mate with a prepared surface of a bone; and bone engaging means extending from said bone facing surface and being adapted to extend into corresponding cavities formed in the prepared surface of the bone when said body is mated with the bone, said bone engaging means comprising one or more of: guiding means adapted to guide the implant into its fixated position with the bone; integration means adapted to promote integration of the implant with the bone; and securing means adapted to secure the implant to the prepared surface of the bone.

### TECHNICAL FIELD

The present invention relates to an orthopaedic implant in particular, but not exclusively, for use in human or animal body in combination with or as a part of an orthopaedic implant system and to a surgical orthopaedic implant system incorporating such an implant.

### BACKGROUND

Any discussion of the background art throughout the specification should in no way be considered as an admission that such background art is prior art, nor that such background art is widely known or forms part of the common general knowledge in the field in Australia or worldwide.

A surgical implant is a medical device placed within or on the surface of the body, intended to replace missing body parts, support organs and tissues, support a bodily function or deliver medication. Implants can be made up of one or more implant components. Implants typically rely on either a press-fit or screw mechanism to lock into position. Press-fit mechanisms rely on friction along the plane of insertion to fix the implant into position. However, they are often susceptible to forces parallel to the axis of insertion which can lead to the implant to loosen.

Orthopaedic implants are medical devices which aim to replace a missing joint or bone to support a damaged bone. Orthopaedic implants tend to be permanent, meaning they are intended to function within a patient for extended periods of time.

Biological tissue must be removed for the implantation of orthopaedic implants, typically resected by mechanical tools such as oscillating saws, burs and drills. In the majority of cases, a significant proportion of healthy tissue is removed to accommodate an implant designed for saws and burs.

A significant proportion of existing joint implants have some number of flat facets on the inner surface to accommodate the flat resections possible with an oscillating saw (see FIG. 1f) or basic curved surfaces with a surgical bur. For example, a typical femoral implant component for a total knee replacement (see FIG. 1d) has five flat facets on the inner (i.e., bone-engaging) surface which are created by five corresponding resections made with an oscillating saw.

The success of an orthopaedic implant is largely determined by the ability for accurate positioning of the implant and the long-term survivorship. Existing means of accurately aligning and positioning an implant rely on accurate bone cutting and surgeon expertise.

The long-term survivorship of an implant relies on primary and secondary fixation. Primary fixation is the short-term fixation of the implant which typically involves mechanical engagement between an implant and the biological tissue. Secondary fixation is medium-long term fixation which involves the integration of the implant with biological tissue. In the case of implants bonding with bone, this is called osseointegration. Secondary fixation is only possible in cementless implants which do not utilise bone cement, since the use of bone cement between the implant and bone creates a barrier and prevents osseointegration.

Orthopaedic implants are prone to high rates of revision surgery as a result in inadequate fixation or mispositioning of the implant. In the case of primary total knee arthroplasty procedures within Australia, revision procedures are required within 5 years for 3.5% of primary total knee replacements. Of these revisions, aseptic loosening, instability and malalignment cause 25%, 8.1% and 2.2% of the revisions respectively. Another 18% of revisions were a result of pain, although it is challenging to identify the point of failure with such symptoms.

Toksvig-Larsen found that assessing the cut surface of tibial plateaus, there was 7-75% of surface contact of an implant with 5 mm of subsidence which could be expected under load. Fixation of a cementless press-fit implant requires a gap of less than 0.3-0.5 mm for strong ingrowth between the bone and implant. This is a contributing factor to the existing survivorship of orthopaedic implants.

It is against this background that the present invention has been developed.

### SUMMARY

The present disclosure relates to an orthopaedic implant for use with or as part of an orthopaedic implant system, comprising: a body having a bone facing surface configured to mate with a prepared surface of a bone; bone engaging means extending from said bone facing surface and being adapted to extend into corresponding cavities formed in the prepared surface of the bone when said body is mated with the bone, said bone engaging means comprising one or more of: guiding means adapted to guide the implant into its fixated position with the bone; integration means adapted to promote integration of the implant with the bone; and securing means adapted to secure the implant to the prepared surface of the bone.

The guiding means may comprise one or more guide projections extending from the bone facing surface, the guide projections being adapted to extend into corresponding guide cavities formed in the prepared surface of the bone. The one or more guide projections may have a substantially uniform width as it extends outwardly from the bone face surface. Additionally, each of the one or more guide projections may have a leading portion adapted to promote insertion of the guide projections into the corresponding guide cavities. The leading portion of each of the guide projections may be tapered to assert with location of the guide projections within the guide cavities.

The integration means may comprise one or more integration projections extending from the bone facing surface, the integration projections being adapted to extend into corresponding integration cavities formed in the prepared surface of the bone. In a representative embodiment of the present invention, the one or more integration projections tapers as it extends outwardly from said bone face surface. In a further representative embodiment of the present invention, the one or more integration projections are three dimensional geometric shapes such as, for example, square or rectangular pyramids. However, it should be appreciated that other three dimensional geometric shapes are also contemplated by the present invention.

The shape of the one or more integration projections may be adapted to maximise contact surface area of the interface between the implant and the prepared surface of the bone.

The one or more integration projections may be adapted to resist transverse movement of the implant relative to the prepared surface of the bone when the body is mated with the bone.

The securing means may comprise one or more securing projections extending from the bone facing surface, the securing projections being adapted to extend into corresponding securing cavities formed in the prepared surface of the bone. The one or more securing projections may be ridges positioned orthogonal to the axis of insertion of the implant and/or parallel to the bone facing surface once the body is mated with the bone. Additionally, the ridges may be adapted to form a substrate deforming joint with the prepared surface of the bone, the substrate deforming joint resulting from the resilient (or partially resilient) deformation of the bone. The substrate deforming joint may be formed when the body is mated with the bone. The geometry of the substrate deforming joint may bias the implant onto the prepared surface of the bone. The projection of the ridges from the bone facing surface may diminish nearer the lateral ends of the implant.

The securing means may be positioned adjacent to or intersecting with the guiding means.

The bone engaging means may be adapted to resist rotational and/or translational forces on the implant once the body is mated with the bone.

The bone facing surface and/or bone engaging means may increase the surface area of the interface between the implant and the prepared surface of the bone by up to 20% relative to an orthopaedic implant with only planar bone facing surfaces. Alternatively, the bone facing surface and/or bone engaging means increases the surface area of the interface between the implant and the prepared surface of the bone by up to 50% relative to an orthopaedic implant with only planar bone facing surfaces. Alternatively, the bone facing surface and/or bone engaging means increases the surface area of the interface between the implant and the prepared surface of the bone by up to 100% relative to an orthopaedic implant with only planar bone facing surfaces. Alternatively, the bone facing surface and/or bone engaging means increases the surface area of the interface between the implant and the prepared surface of the bone by up to 1000% relative to an orthopaedic implant with only planar bone facing surfaces.

The bone facing surface of the body may be configured to mate with the prepared surface of a femoral bone. Alternatively, the bone facing surface of the body may be configured to mate with the prepared surface of a tibial bone. Alternatively, the bone facing surface of the body may be configured to mate with the prepared surface of a patella bone.

The present disclosure also relates to a surgical orthopaedic implant system including the orthopaedic implant the orthopaedic implant described above, the system comprising the steps of: (a) gaining access to an implant area within a patient's body; (b) preparing an implant receiving surface of a bone within the implant area to receive and mate with the bone facing surface; and (c) securing the orthopaedic implant onto the implant receiving surface of the bone.

The step of preparing the implant receiving surface may further comprise the step of creating corresponding cavities on the implant receiving surface, the corresponding cavities being configured to mate with and substantially engage the bone engaging means when the body is mated with the bone.

The step of creating corresponding cavities on the implant receiving surface may involve the use of a laser bone ablation device.

### BRIEF DESCRIPTION OF THE DRAWINGS

Embodiments of the present invention will now be described with reference to the accompanying drawings. These embodiments are given by way of illustration only and other embodiments of the invention are also possible. Consequently, the particularity of the accompanying drawings is not to be understood as superseding the generality of the preceding description. In the drawings:

FIG. 1a is a perspective view of a femoral component in accordance with a representative embodiment of the present invention;

FIGS. 1b-1f are top, side, bottom, back, and front views, respectively, of the femoral component of FIG. 1a;

FIG. 1g is the sectional view D-D of the femoral component of FIG. 1b;

FIG. 1h is the detail view E of the femoral component of FIG. 1g;

FIG. 1i is the detail view A of the femoral component of FIG. 1e;

FIG. 1j is the sectional view B-B of the femoral component of FIG. 1e;

FIG. 1k is the sectional view C-C of the femoral component of FIG. 1c;

FIGS. 2a and 2b are front and back perspective views, respectively, of a femur with matching resections in accordance with the femoral component of FIG. 1a;

FIGS. 2c-2f are front, side, top, and bottom views, respectively, of the femur of FIG. 2a;

FIG. 3a is a perspective view of a femoral component of a conventional total knee replacement implant;

FIGS. 3b-d are front, back, and side views, respectively, of the femoral component of FIG. 3a;

FIG. 3e is a front view of the current state-of-the-art resections applied to a distal femoral bone to fit the femoral component of FIG. 3a;

FIG. 3g is a perspective view of the femoral component of FIG. 3a in position for insertion onto the distal femoral bone of FIG. 3e;

FIGS. 4a and 4b are front and back perspective views, respectively, of a femoral component in accordance with a representative embodiment of the present invention;

FIGS. 4c-4f are top, side, front, and back views, respectively, of the femoral component of FIG. 4a;

FIGS. 4g-4i are sectional views A-A, B-B, and C-C, respectively, of the femoral component of FIG. 4d;

FIGS. 5a-5c are top, side, and front views, respectively, of a guide in accordance with a representative embodiment of the present invention;

FIGS. 5d-5f are top, side, and front views, respectively, of the guide of FIG. 5a;

FIG. 5g is a front view of an alternative embodiment of the guide of FIG. 5f;

FIGS. 6a-d are variations of the guide in accordance with representative embodiments of the present invention;

FIG. 7 is a guide with matching void in accordance with a representative embodiment of the present invention;

FIG. 8 is a top view of guides with matching voids in accordance with a representative embodiment of the present invention;

FIG. 9a is a perspective view of integrating features in accordance with a representative embodiment of the present invention;

FIGS. 9b-9d are top, side, and front views, respectively, of the integrating features of FIG. 9a;

FIG. 9e is a sectional view A-A of the integrating features of FIG. 9c;

FIG. 10 is a side diagrammatic view of a series of ridges in accordance with a representative embodiment of the present invention;

FIG. 11a is a perspective view of a series of ridges in accordance with a representative embodiment of the present invention;

FIGS. 11b-11d are top, side, and front views, respectively, of the series of ridges of FIG. 11a;

FIGS. 11e-11h are sectional views A-A, B-B, C-C, and D-D, respectively, of the series of ridges of FIG. 11;

FIG. 12a is a perspective view of a series of ridges in accordance with a representative embodiment of the present invention;

FIGS. 12b-12d are top, side, and front views, respectively, of the series of ridges of FIG. 12a;

FIGS. 12e-12h are sectional views A-A, B-B, C-C, and D-D, respectively, of the series of ridges of FIG. 12b;

FIG. 13a is a perspective view of a series of ridges in accordance with a representative embodiment of the present invention;

FIGS. 13b-13d are top, side, and front views, respectively, of the series of ridges of FIG. 13a;

FIGS. 13e-13g are sectional views A-A, B-B, and C-C, respectively, of the series of ridges of FIG. 13b;

FIG. 14a is a perspective view of a series of ridges in accordance with a representative embodiment of the present invention;

FIGS. 14b-14d are top, side, and front views, respectively, of the series of ridges of FIG. 14a;

FIGS. 14e-14g are sectional views A-A, B-B, and C-C, respectively, of the series of ridges of FIG. 14b;

FIG. 15a is a perspective view of a series of ridges in accordance with a representative embodiment of the present invention;

FIG. 15b is a top view of the series of ridges of FIG. 15a;

FIG. 15c is a sectional view A-A of the series of ridges of FIG. 15b;

FIG. 16a is a perspective view of a series of ridges with guides in accordance with a representative embodiment of the present invention;

FIGS. 16b-16d are top, side, and front views, respectively, of the series of ridges with guides of FIG. 16a;

FIG. 16e is a sectional view A-A of the series of ridges of FIG. 16b;

FIG. 17a is a perspective view of a series of ridges with guides in accordance with a representative embodiment of the present invention;

FIGS. 17b-17d are top, side, and front views, respectively, of the series of ridges with guides of FIG. 17a;

FIG. 18a is a perspective view of integrating and securing features in accordance with a representative embodiment of the present invention;

FIGS. 18b-18d are top, side, and front views, respectively, of the integrating and securing features of FIG. 18a;

FIGS. 18e and 18f are sectional views A-A and B-B, respectively, of the integrating and securing features of FIG. 18c;

FIG. 18g is the detail view C of the integrating and securing features of FIG. 18c;

FIG. 19a is a perspective view of integrating and securing features in accordance with a representative embodiment of the present invention;

FIGS. 19b-19d are top, side, and front views, respectively, of the integrating and securing features of FIG. 19a;

FIG. 19e is a sectional view A-A of the integrating and securing features of FIG. 19c;

FIG. 19f is the detail view B of the integrating and securing features of FIG. 19c;

FIG. 20a is a perspective view of truncated securing features in accordance with a representative embodiment of the present invention;

FIGS. 20b-20d are top, side, and front views, respectively, of the truncated securing features of FIG. 20a;

FIG. 20e is a sectional view A-A of the truncated securing features of FIG. 20c;

FIG. 21a is a perspective view of a truncated securing feature in accordance with a representative embodiment of the present invention;

FIG. 21b is a perspective view of the full securing feature of FIG. 21a;

FIGS. 22a-22c are back views of various compatible sizes of femoral components in accordance with a representative embodiment of the present invention;

FIGS. 23a and 23b are perspective views of the femoral component in accordance with a representative embodiment of the present invention;

FIGS. 23c-23f are top, bottom side, and back views of the femoral component of FIG. 23a;

FIG. 23g is a sectional view A-A of the femoral component of FIG. 23e;

FIGS. 23h and 23i are sectional views B-B and C-C, respectively, of the femoral component of FIG. 23f;

FIGS. 24a and 24b are perspective views of a femoral component in accordance with a representative embodiment of the present invention;

FIGS. 24c-24f are top, bottom, side, and back views, respectively, of the femoral component of FIG. 24a;

FIGS. 24g and 24i are sectional views A-A, B-B and C-C, respectively, of the femoral component of FIG. 24f;

FIGS. 25a and 25b are front and back perspective views, respectively, of a femur with matching resections in accordance with the femoral component of FIG. 24a;

FIGS. 25c-25f are front, side, top, and bottom views, respectively, of the femur of FIG. 25a;

FIGS. 26a and 26b are perspective views of a femoral component in accordance with a representative embodiment of the present invention;

FIGS. 26c-26f are top, bottom, side, and back views, respectively, of the femoral component of FIG. 26a;

FIGS. 26g and 26i are sectional views B-B and C-C, respectively, of the femoral component of FIG. 26f;

FIGS. 27a and 27b are front and back perspective views, respectively, of a femur with matching resections in accordance with the femoral component of FIG. 26a;

FIGS. 27c-27f are front, side, top, and bottom views, respectively, of the femur of FIG. 27a;

FIGS. 28a and 28b are perspective views of a femoral component in accordance with a representative embodiment of the present invention;

FIGS. 28c-28f are side, back, top and bottom views, respectively, of the femoral component of FIG. 28a;

FIGS. 28g and 28h are sectional views A-A and B-B, respectively, of the femoral component of FIG. 28c;

FIG. 28i is a sectional view C-C of the femoral component of FIG. 28d;

FIGS. 29a and 29b are perspective views of a femur with matching resections in accordance with the femoral component of FIG. 28a;

FIGS. 29c-29f are front, side, top, and bottom views, respectively, of the femur of FIG. 29a;

FIGS. 30a and 30b are perspective views of a femoral component in accordance with a representative embodiment of the present invention;

FIGS. 30c-30f are top, front, bottom, side, and back views, respectively, of the femoral component of FIG. 30a;

FIGS. 31a and 31b are perspective views of a femur with matching resections in accordance with the femoral component of FIG. 30a;

FIGS. 31c-31f are front, side, top, and bottom views, respectively, of the femur of FIG. 31a;

FIGS. 32a and 32b are perspective views of a femoral component in accordance with a representative embodiment of the present invention;

FIGS. 32c-32f are top, side, bottom, and back views, respectively, of the femoral component of FIG. 32a;

FIGS. 32g-32j are sectional views A-A, B-B, C-C, and D-D, respectively, of the femoral component of FIG. 32f;

FIGS. 33a and 33b are perspective views of a femur with matching resections in accordance with the femoral component of FIG. 32a;

FIGS. 33c-33f are front, side, top, and bottom views, respectively, of the femur of FIG. 33a;

FIGS. 34a and 34b are perspective views of a femoral component in accordance with a representative embodiment of the present invention;

FIGS. 34c-34f are front, side, top, and bottom views, respectively, of the femoral component of FIG. 34a;

FIGS. 35a and 35b are perspective views of a femoral component with a femur with matching resections in accordance with a representative embodiment of the present invention;

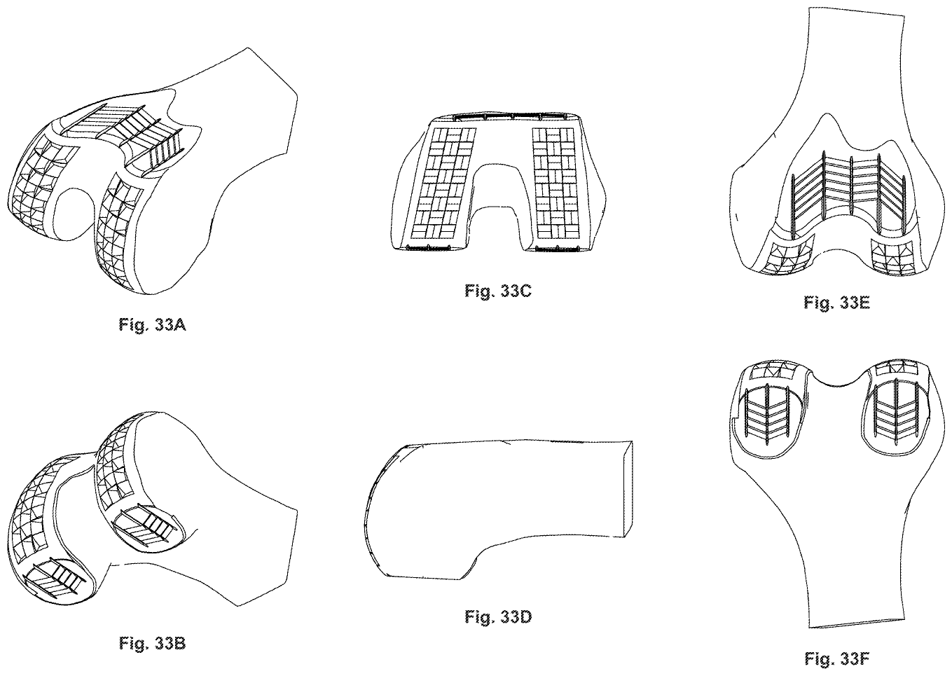

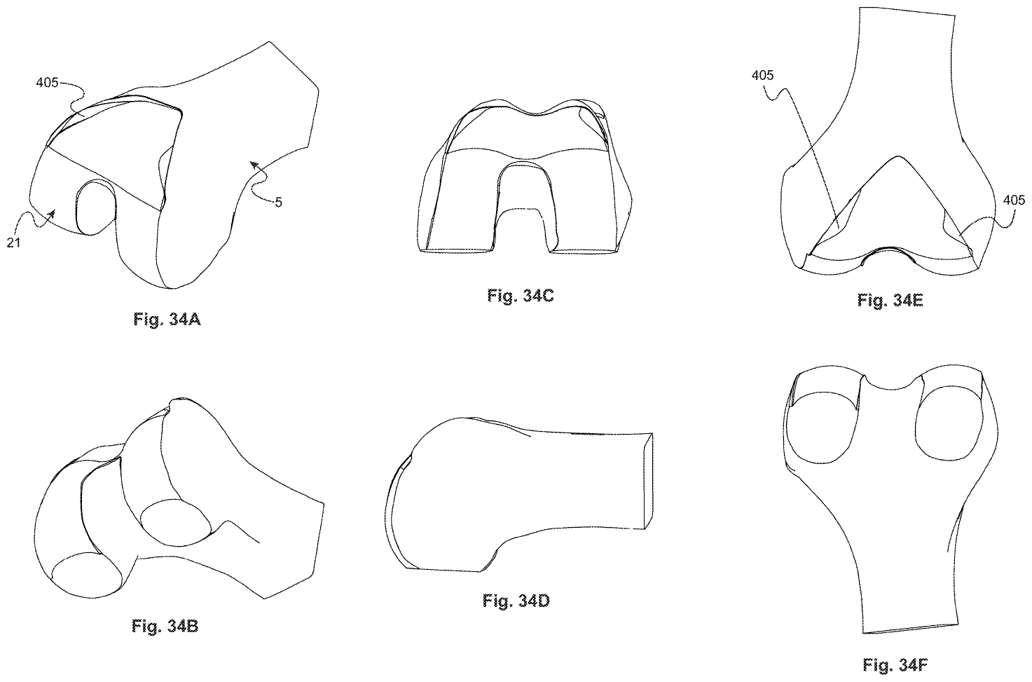

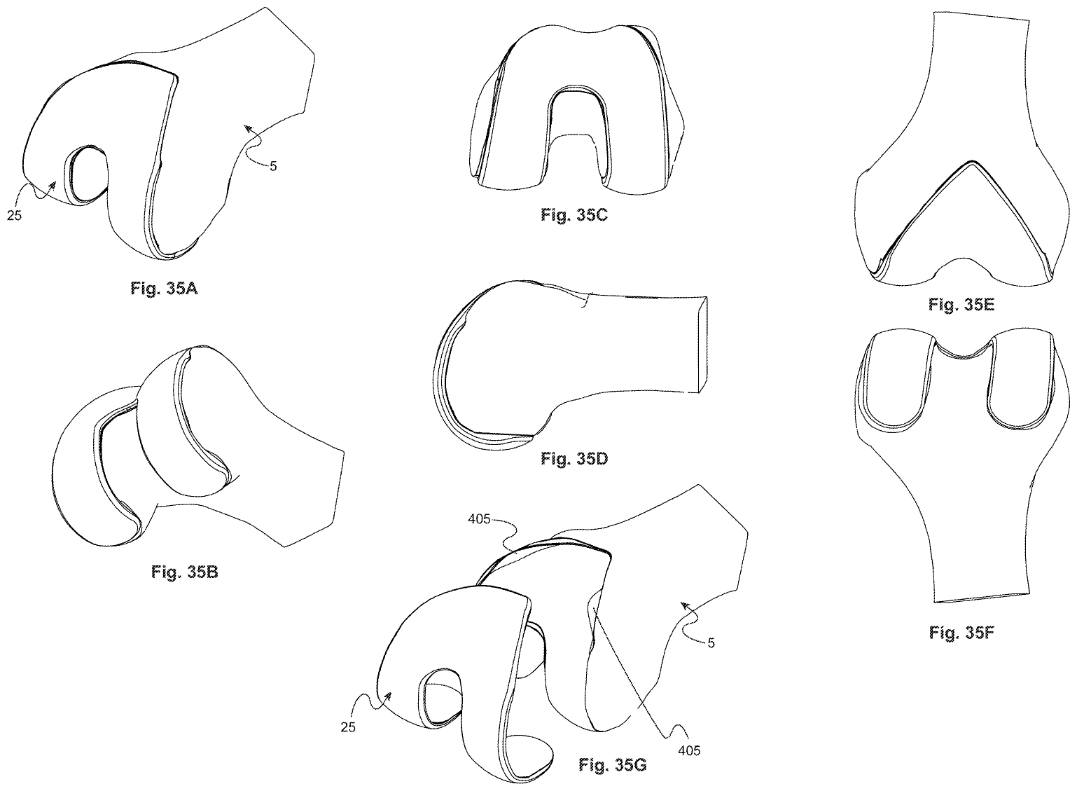

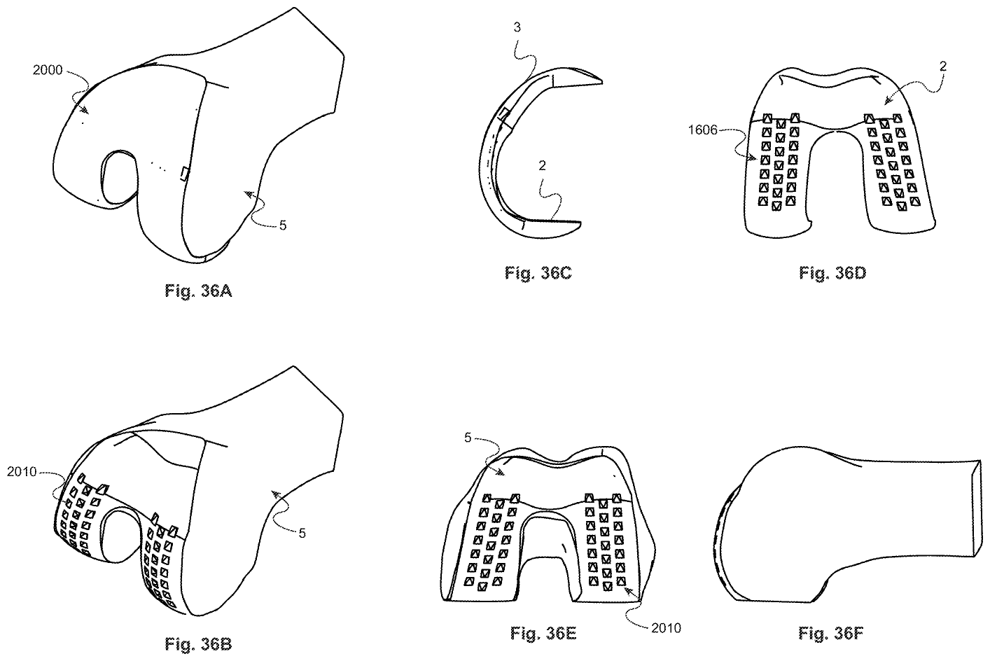

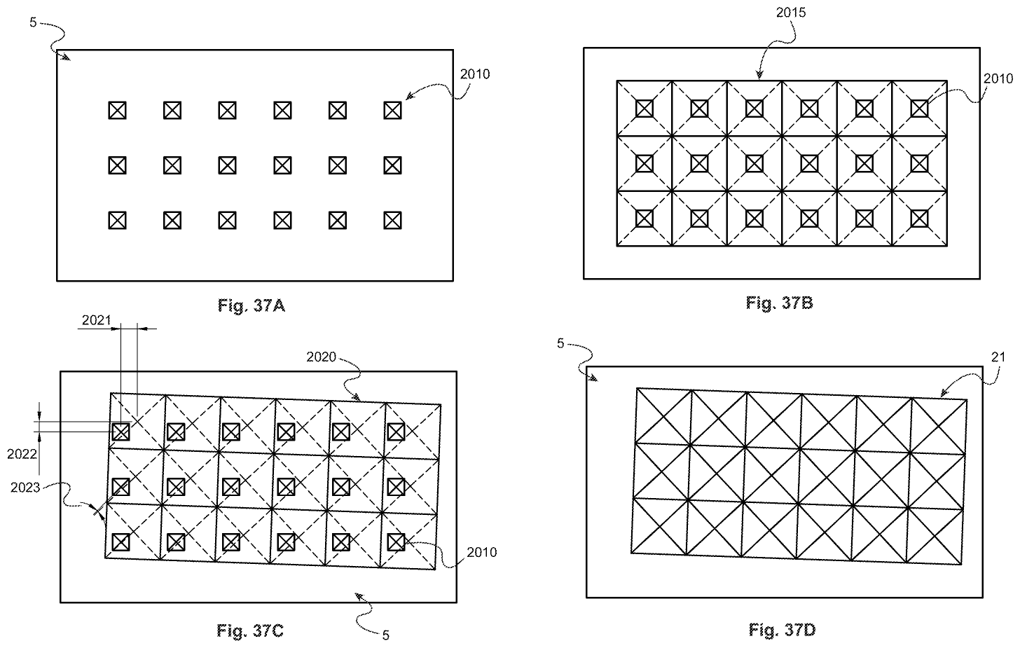

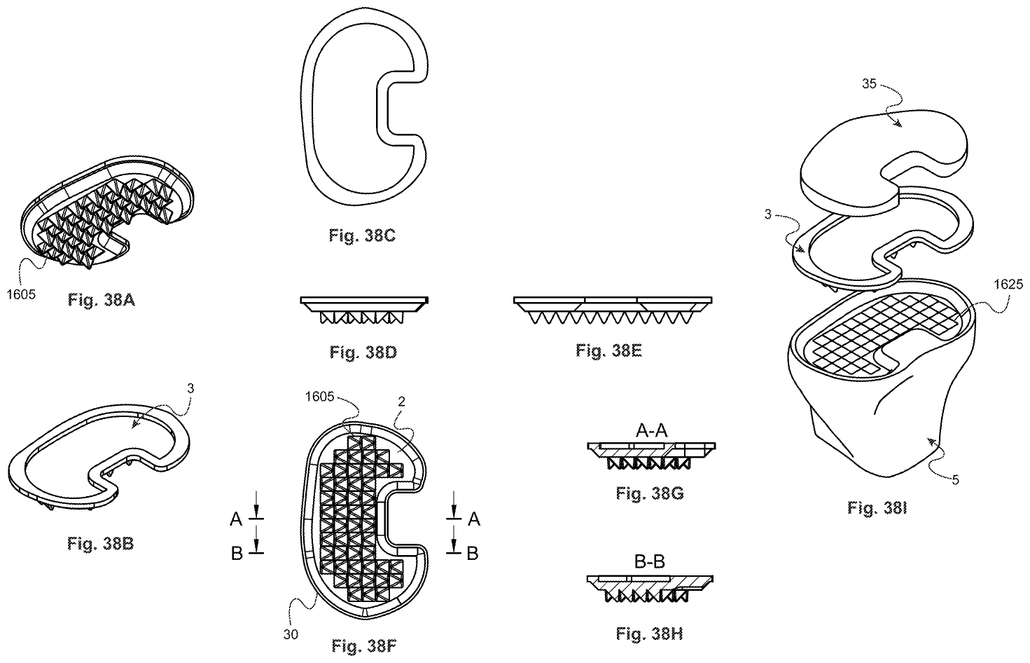

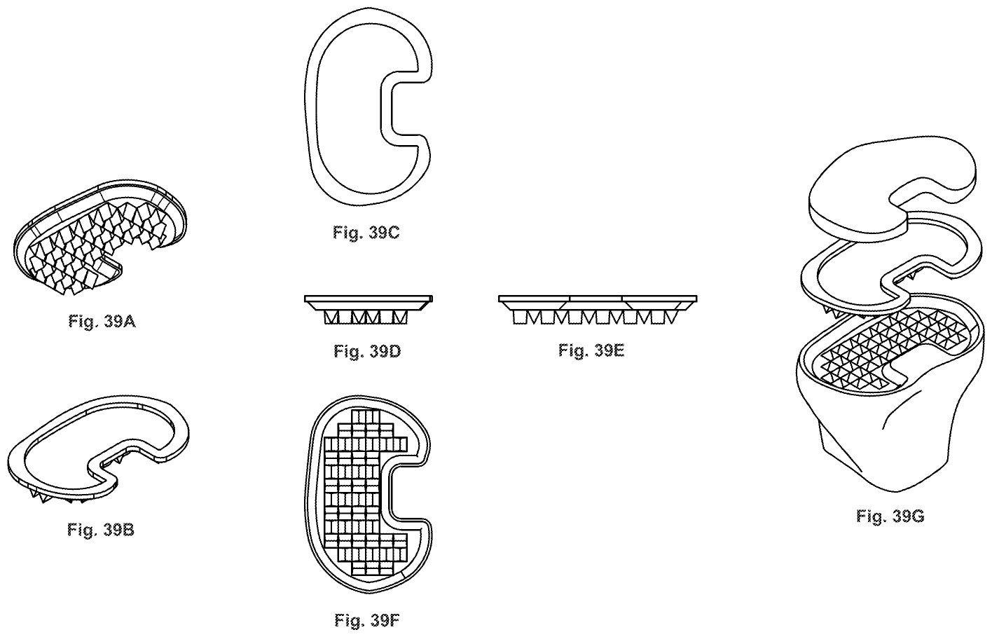

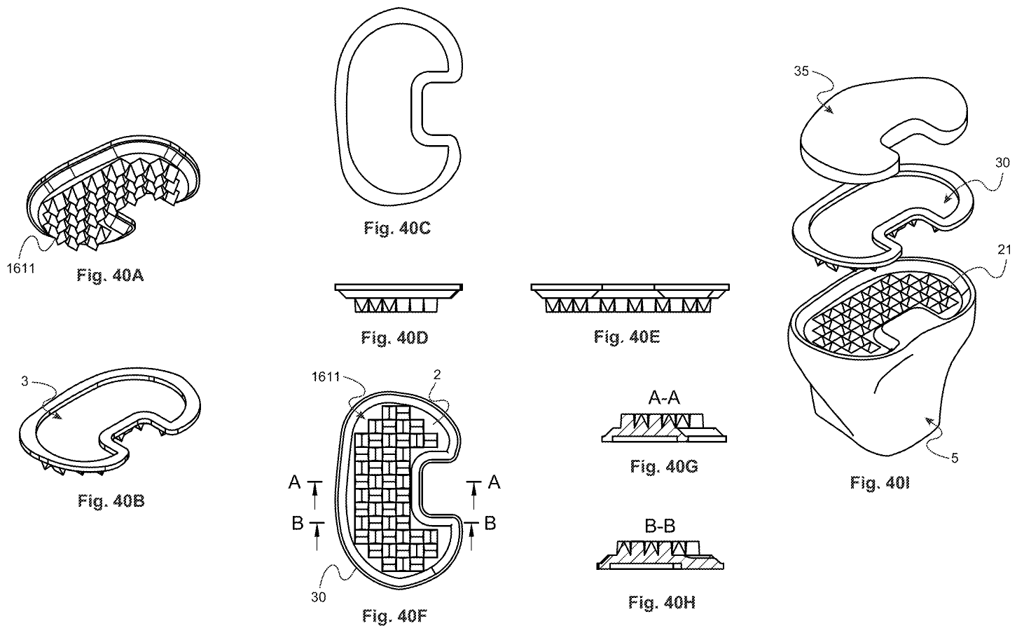

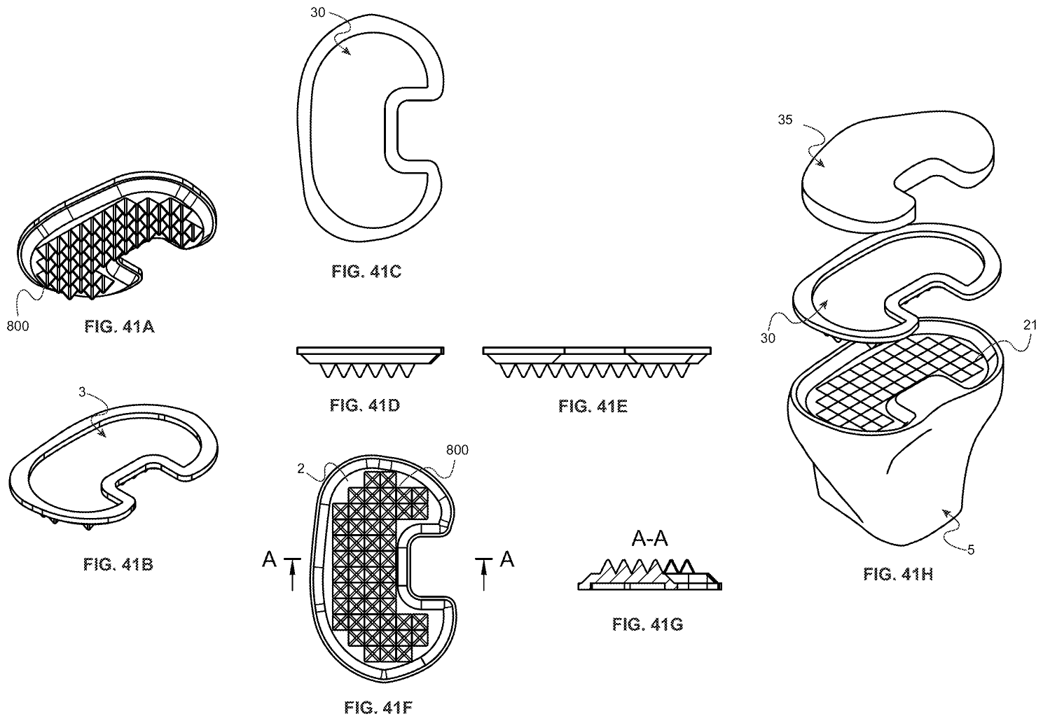

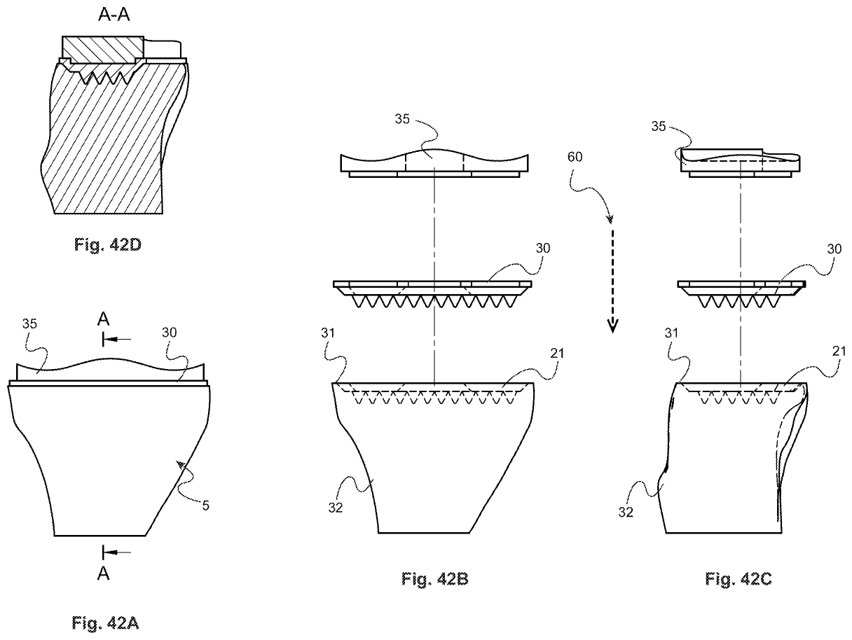

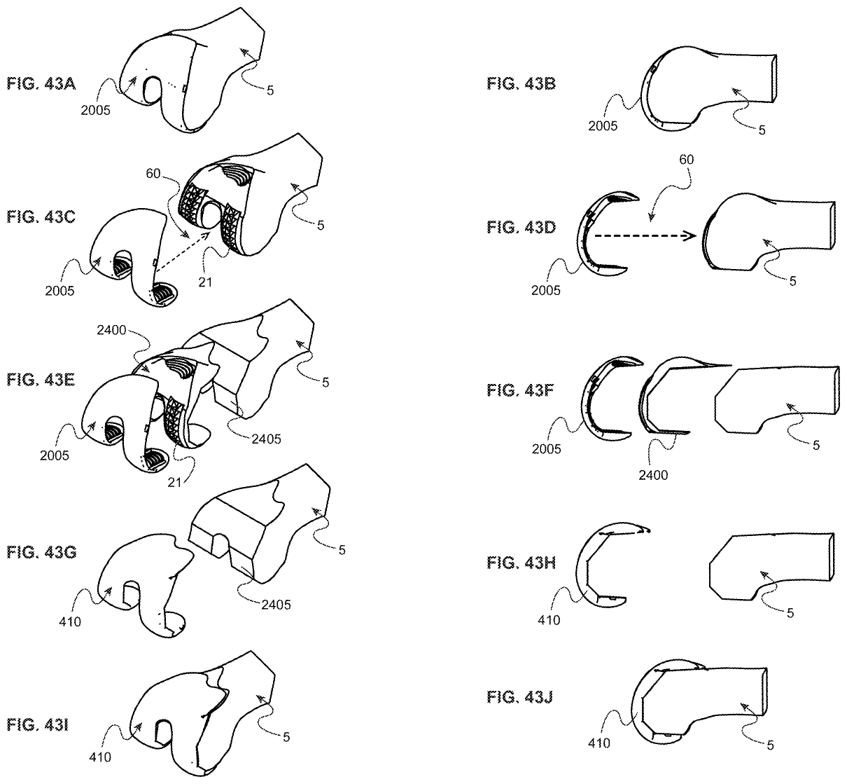

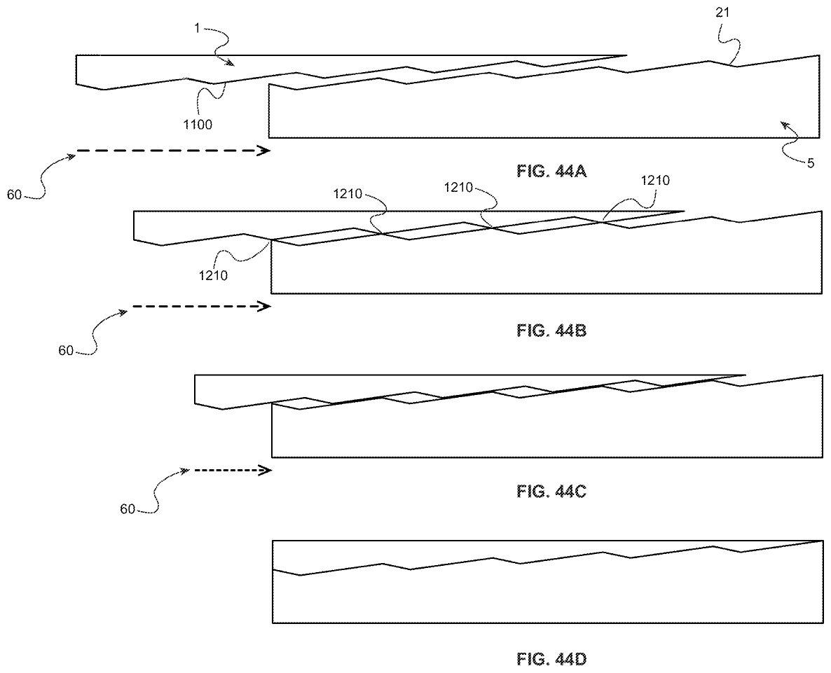

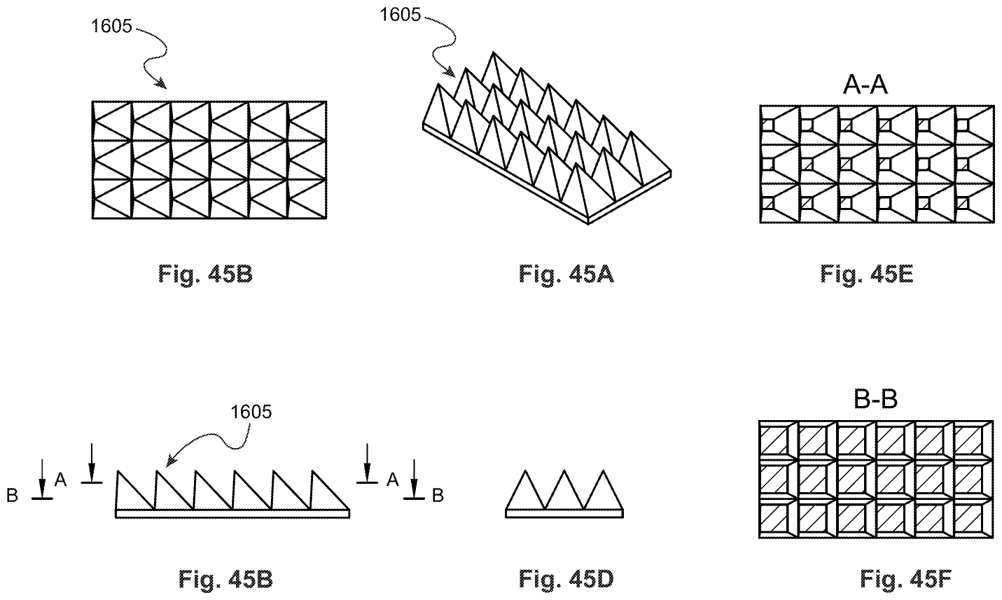

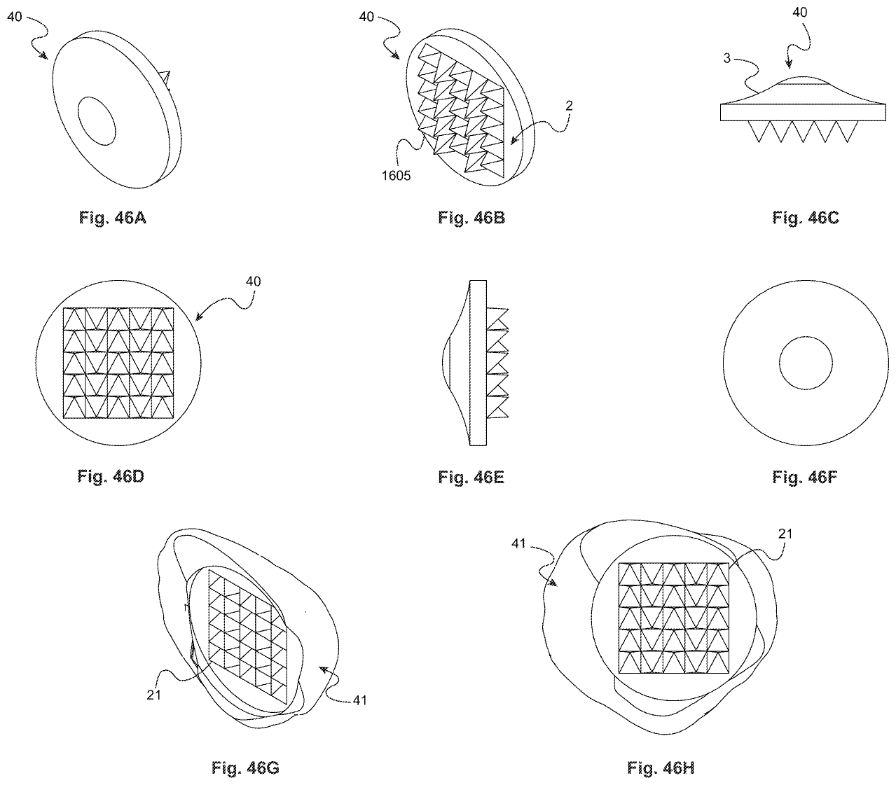

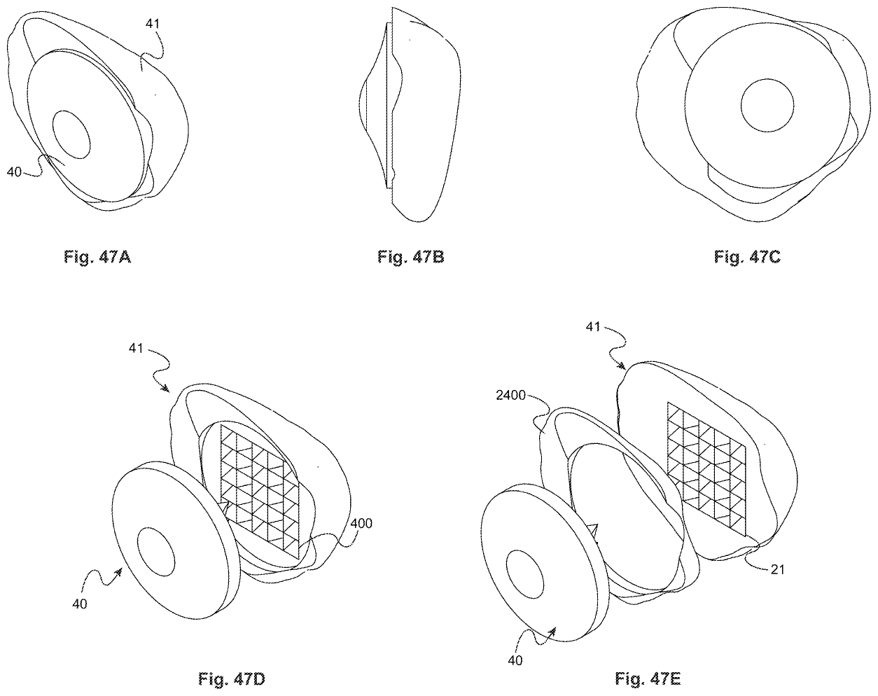

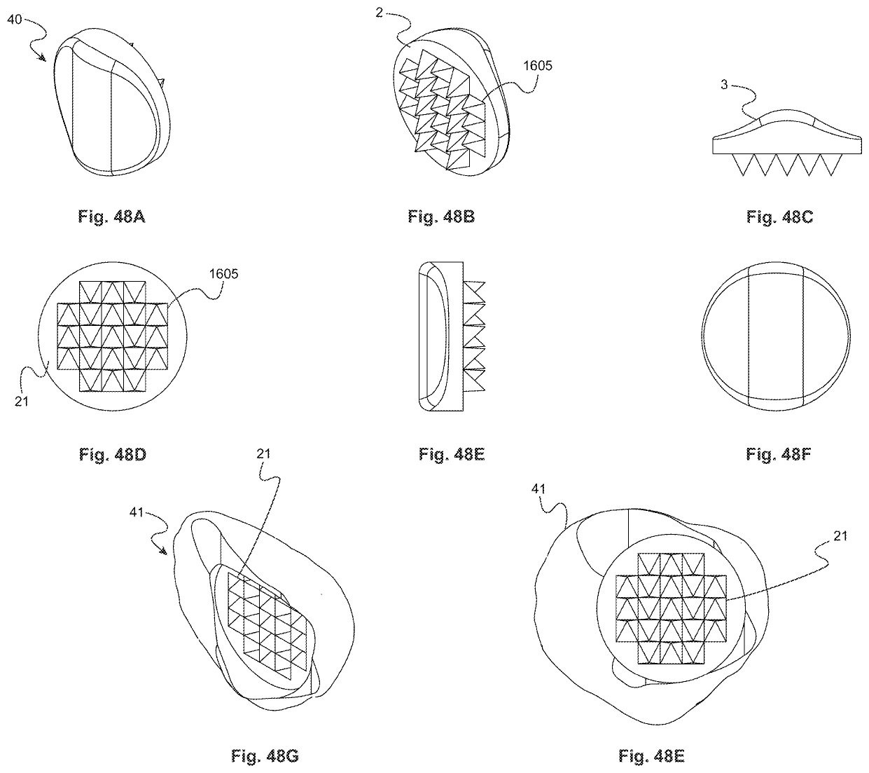

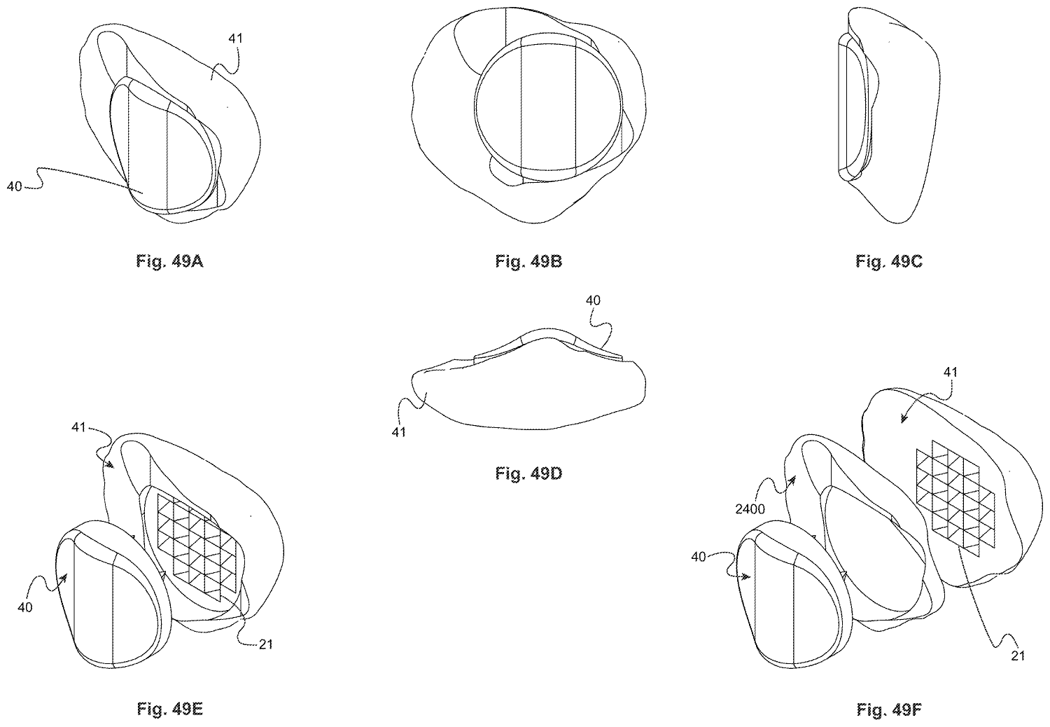

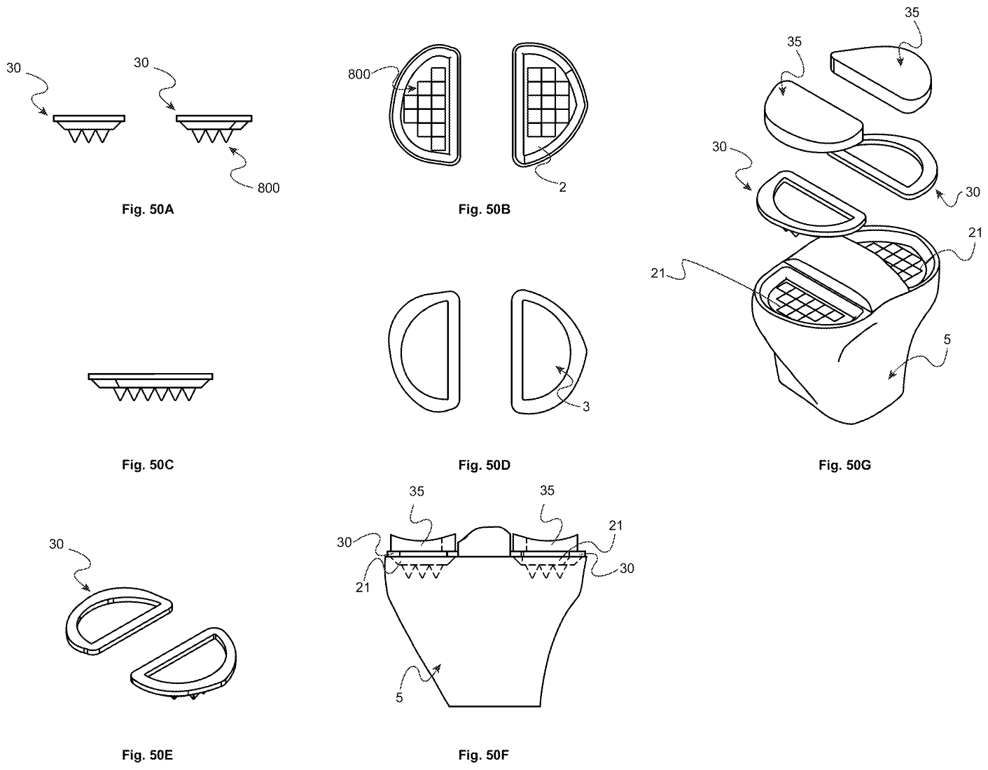

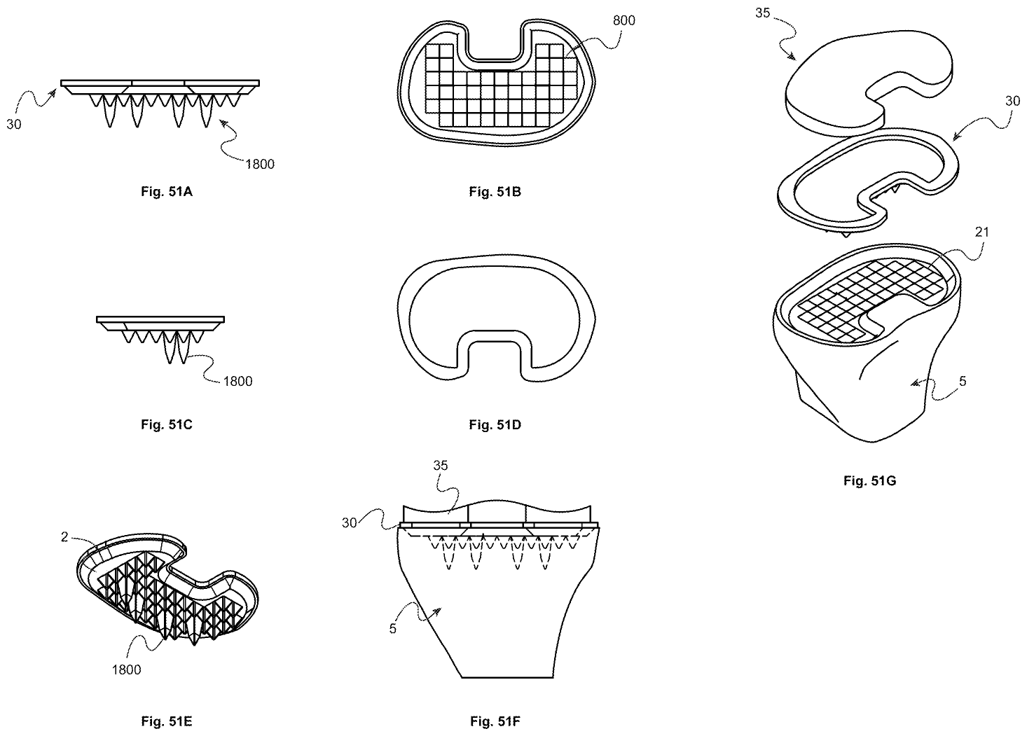

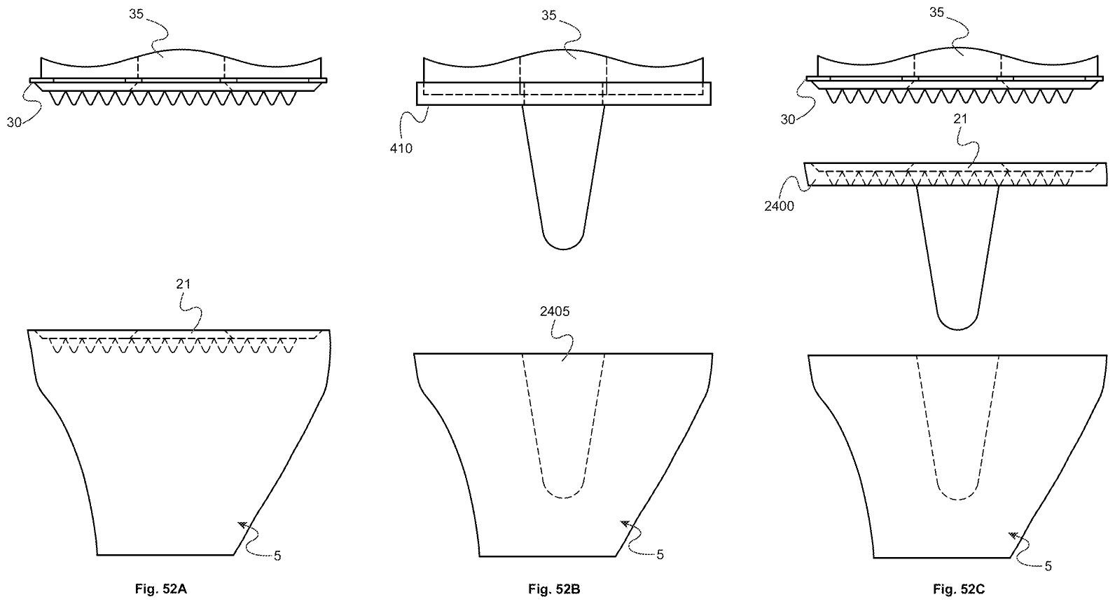

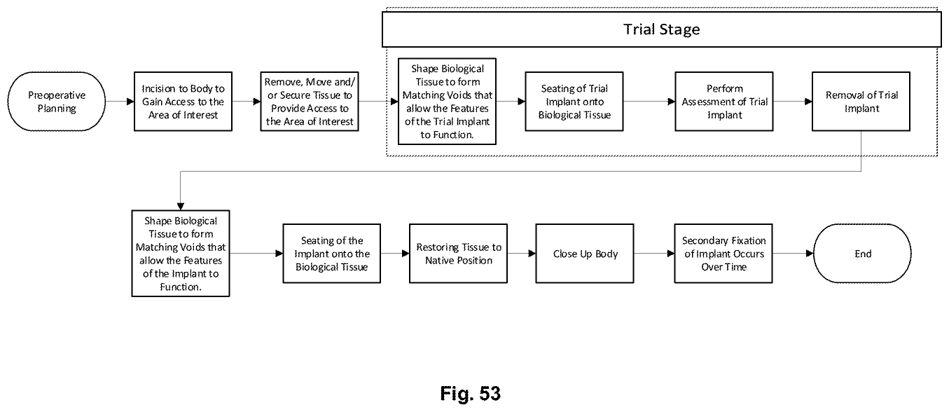

FIGS. 33c-33f are front, side, top, and bottom views, respectively, of the femur of FIG. 35a;

FIG. 35g is a perspective exploded view of the femoral component and femur of FIG. 35a;

FIG. 36a is a perspective views of a trial femoral component with a femur with matching resections in accordance with a representative embodiment of the present invention;

FIG. 36b is a perspective view of the femur with matching resections to the inner surface features of the trial femoral component of FIG. 36a;

FIGS. 36c and 36d are side and back views, respectively, of the trial femoral component of FIG. 36a;

FIGS. 36e and 36f are front and side views, respectively, of the femur of FIG. 36a;

FIG. 37a is a top view of trial voids in accordance with a representative embodiment of the present invention;

FIG. 37b is a top view of trial voids of FIG. 37a with proposed voids overlaid;

FIG. 37c is a top view of trial voids of FIG. 37a with adjusted proposed voids overlaid;

FIG. 37d is a top view of resected matching voids;

FIGS. 38a and 38b are perspective views of a tibial component in accordance with a representative embodiment of the present invention;

FIGS. 38c-38f are top, side, front, and bottom views, respectively, of the tibial component of FIG. 38a;

FIGS. 38g and 38h are sectional views A-A and B-B, respectively, of the tibial component of FIG. 38f;

FIG. 38i is a perspective exploded view of the tibial component of FIG. 38a;

FIGS. 39a and 39b are perspective views of a tibial component in accordance with a representative embodiment of the present invention;

FIGS. 39c-39f are top, side, front, and bottom views, respectively, of the tibial component of FIG. 39a;

FIG. 39g is a perspective exploded view of the tibial component of FIG. 39a;

FIGS. 40a and 40b are perspective views of a tibial component in accordance with a representative embodiment of the present invention;

FIGS. 40c-40f are top, side, front, and bottom views, respectively, of the tibial component of FIG. 40a;

FIGS. 40g and 40h are sectional views A-A and B-B, respectively, of the tibial component of FIG. 40f;

FIG. 40i is a perspective exploded view of the tibial component of FIG. 40a;

FIGS. 41a and 41b are perspective views of a tibial component in accordance with a representative embodiment of the present invention;

FIGS. 41c-41f are top, side, front, and bottom views, respectively, of the tibial component of FIG. 41a;

FIG. 41g is a sectional view A-A of the tibial component of FIG. 41f;

FIG. 41h is a perspective exploded view of the tibial component of FIG. 41a;

FIG. 42a is a front view of a tibial implant seated in a tibia in accordance with a representative embodiment of the present invention;

FIGS. 42b and 42c are front and side exploded views, respectively, of the tibial implant and tibia of FIG. 42a;

FIG. 42d is a sectional view A-A of the tibial implant and tibia of FIG. 42a;

FIGS. 43a and 43b are perspective and side views, respectively, of a femoral component seated on a femur with matching resections in accordance with a representative embodiment of the present invention;

FIGS. 43c and 43d are perspective and side exploded views, respectively, of FIG. 43a and FIG. 43b;

FIGS. 43e and 43f are perspective and side exploded views, respectively, of a femoral component in accordance with a representative embodiment of the present invention, the spared biological tissue, and a femur with conventional resections;

FIGS. 43g and 43h are perspective and side exploded views, respectively, of a conventional femoral component and a femur with conventional resections; and

FIGS. 43i and 43j are perspective and side views, respectively, or the conventional femoral component and a femur with conventional resections of FIGS. 43g and 43h;

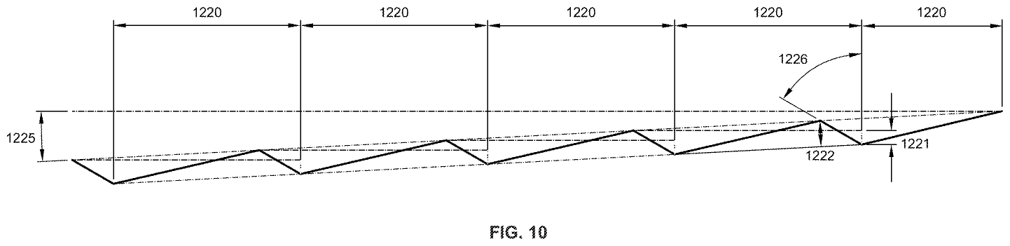

FIGS. 44a-44d are side views of ridges depicted FIGS. 10, visualising the approach of the ridges onto the matching voids 21 and the engagement and formation of the substrate deforming joint 1200;

FIG. 45a is a perspective view of integrating features in accordance with a representative embodiment of the present invention;

FIGS. 45b-45d are top, side, and front views, respectively, of the integrating features of FIG. 45a;

FIGS. 45e and 45f are sectional views A-A and B-B, respectively, of the integrating features of FIG. 45c;

FIGS. 46a-46b are perspective views of a patella component in accordance with a representative embodiment of the present invention;

FIGS. 46c-46f are top, back, side and front views, respectively, of the patella component of FIG. 46a;

FIG. 46g-46h are perspective and front views, respectively, of a patella with matching voids in accordance with the patella component of FIG. 46a;

FIG. 47a-47c are perspective, side and front views, respectively, of a patella component seated on a patella with matching voids in accordance with the patella component of FIG. 46a and patella of FIG. 46g; and

FIG. 47d is a perspective exploded view of the patella component and patella of FIG. 47a.

FIG. 47e is a perspective exploded view of the patella component, spared biological tissue and patella of FIG. 47a.

FIGS. 48a-48b are perspective views of a patella component in accordance with a representative embodiment of the present invention;

FIGS. 48c-48f are top, back, side and front views, respectively, of the patella component of FIG. 48a;

FIG. 48g-48h are perspective and front views, respectively, of a patella with matching voids in accordance with the patella component of FIG. 46a;

FIG. 49a-49d are perspective, front, side and bottom views, respectively, of a patella component seated on a patella with matching voids in accordance with the patella component of FIG. 48a and patella of FIG. 48g; and

FIG. 49e is a perspective exploded view of the patella component and patella of FIG. 49a.

FIG. 49f is a perspective exploded view of the patella component, spared biological tissue and patella of FIG. 49a.

FIGS. 50a-50d are front, bottom, side and top views, respectively, of the double uni-compartmental tibial component;

FIG. 50e is a perspective view of the double uni-compartmental tibial component of FIG. 50a;

FIG. 50f is a front view of the tibial component of FIG. 50a in its final seated position in the tibia.

FIG. 50g is an exploded perspective view of the spacer, tibial component of FIG. 50a and biological tissue with corresponding matching voids.

FIGS. 51a-51d are front, bottom, side and top views, respectively, of a tibial component in accordance with a representative embodiment of the present invention;

FIG. 51e is a perspective view of the tibial component of FIG. 50a.

FIG. 51f is a front view of the tibial component of FIG. 51a in its final seated position in the tibia.

FIG. 51g is an explode perspective view of the spacer, tibial component of FIG. 51a and biological tissue with corresponding matching voids.

FIG. 52a is an exploded front view of a representative embodiment of the present invention, a tibial implant, and the respective biological tissue with matching voids.

FIG. 52b is an exploded front view of a representative embodiment of a conventional implant and the respective biological tissue with matching voids.

FIG. 52c is an exploded front view of a representative embodiment of the present invention, a tibial implant, the spared biological tissue and the respective biological tissue with matching voids for a conventional implant.

FIG. 53 is a flowchart summarising the steps involved in a representative embodiment of a surgical orthopaedic system in accordance with the present invention.

### DETAILED DESCRIPTION

The following detailed description is an exemplification of the invention and should not be limited in scope by the embodiments depicted nor should it be understood in any way as a restriction on the broad description of the invention as set out hereinbefore. These embodiments are described in sufficient detail to allow those skilled in the art to practise or exercise the invention. The precise shape, size and appearance of the components described or illustrated are not expected of nor required from the invention unless stated otherwise. It is to be understood that any utilisation combination or structural, logical, electrical and mechanical changes, variations, augmentations or modifications to any of the mentioned or otherwise related embodiments may be made without departing from the scope of the invention. Similarly, any functionally equivalent products, compositions and methods will also remain within this scope along with all singular, combination and sequences of steps, features, structures, sequences, processes, combinations and compounds referred to or indicated within this description either singularly or collectively.

The entire disclosure of all documentation including patents, patent applications, journal articles, laboratory manuals, books, charts, repositories, and any other form of documentation or otherwise referenced resources cited herein is by no means an admission of prior art, prior or common knowledge required by those skilled in the art or any other connections or assumptions towards the invention unless mentioned otherwise.

Throughout this description, unless stated otherwise, the words ‘comprise’, ‘include’ and any variations which may consist of ‘comprising’, ‘comprises’, ‘including’ or ‘includes’ will be understood to imply the inclusion of a stated integer or a group of integers but not the exclusion of any other integer or group of integers.

An orthopaedic implant is a medical device which replaces, supports or enhances a biological structure such as, for example, bone in a human or animal body. An orthopaedic implant in accordance with a representative embodiment of the present invention comprises a body 1 having a bone facing surface 2 configured to mate with a prepared surface of a bone 5.

The implant body 1 includes bone engaging means extending from the bone facing surface 2 and being adapted to extend into corresponding cavities formed in the prepared surface of the bone 5 when the body 1 is mated with the bone 5. The bone engaging means comprising a guiding means adapted to guide the implant 1 into its fixated position with the bone 5, integration means adapted to promote integration of the implant 1 with the bone 5; and securing means adapted to secure the implant 1 to the prepared surface of the bone 5.

The outer surface 3 of an implant is where the shape relates to the function of the implant 1. This is particularly relevant in the context of orthopaedic implants.

An implant 1 can be configured through variations in geometric features which are applied to the inner surface 2 of an implant 1. An inner surface geometric feature 9 is a physical shape, protrusion or void present on the implant 1.

An inner surface geometric feature 9 can be of any scale which aids in the implantation and survivorship of an implant 1. The inner surface geometric features 9 depicted throughout this patent are one representation, however the inner surface geometric features 9 could span from a scale of 1 mm to 5 cm dependent on the implant 1 in question.

Matching voids 21 are voids on the biological tissue 5 matching, correlating or corresponding to the inner surface geometric features 9 on the implant 1 as a result of resections. Matching voids 21 on the biological tissue 5 are created through accurate and precise biological tissue 5 shaping referred to as matching resections 20.

The combination of inner surface geometric features 9 and matching voids 21 can create various benefits which aid the survivorship and accurate positioning of the implant 1. Negating the impact of implant 1 alignment and positioning, the survivorship of the implant 1 is dependent on the degree of primary fixation 45 and secondary fixation 50. Primary fixation 45 is the immediate fixation of an implant 1, typically due to mechanical engagement, between the implant 1 and biological tissue 5. Secondary fixation 50 is the medium to long-term fixation of an implant 1, typically due to osseointegration, between the implant 1 and biological tissue 5.

Inner surface geometric features 9 which help accurately align the implant 1 and guide it into its final seated position are referred to as guiding features 10. Inner surface geometric features 9 which aim to secure the implant to the biological tissue, providing a means of primary fixation 45 are referred to as securing features 12. Inner surface geometric features 9 which aid in the secondary fixation 50 of the implant and aid integration are referred to as integrating features 11.

Guiding features 10, integrating features 11, and securing features 12 are combined to form the inner surface 2 of an implant 1. The configuration, arrangement, and combination of different types of features provides a means of assuring survivorship and accurate positioning of the implant 1. Each specific combination of inner surface geometric features 9 has a set of matching voids 21 created with accurate resections that allow the secure seating of the implant 1.

The combination of inner surface geometric features 9 and the inner and outer surface design of an implant can be configured to preserve biological tissue 5 through the minimisation of biological tissue required to be resected to produce the matching voids to seat the implant to the biological tissue.

Orthopaedic implants currently have issues with alignment and positioning of the implant 1 and aseptic loosening. This is particularly prevalent in primary total knee arthroplasty implants. Creating an inner and outer surface design which incorporate inner surface geometric features 9 aims to resolve these issues.

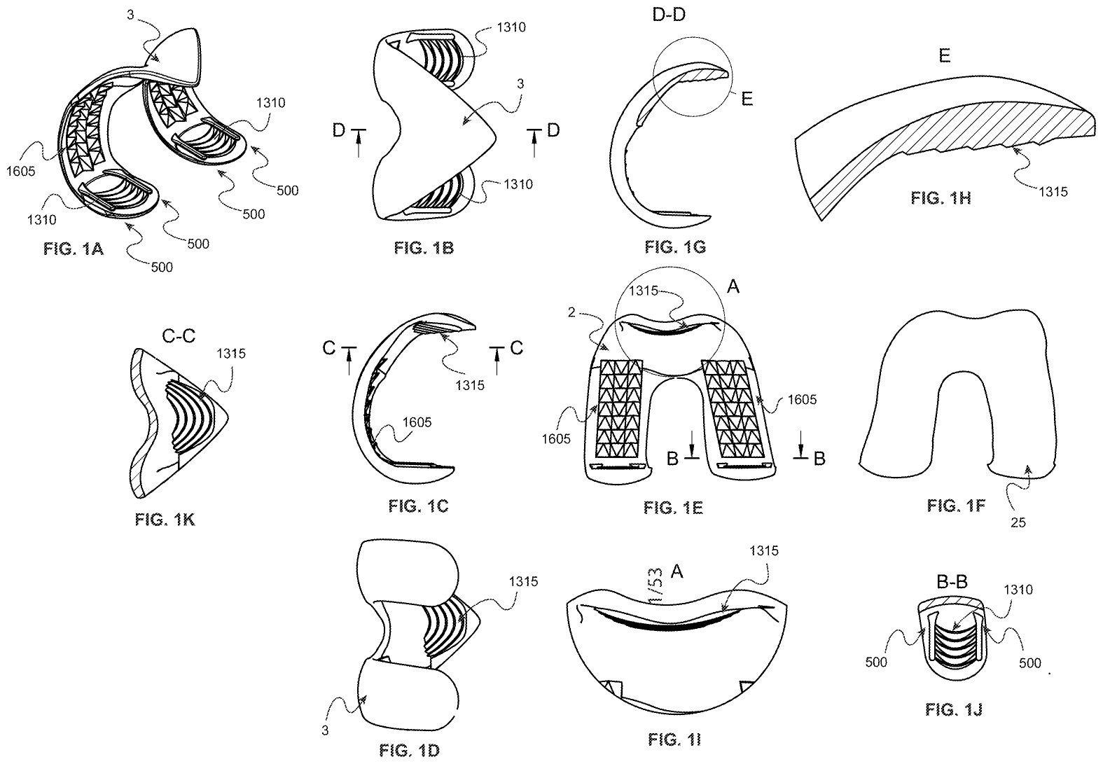

In the preferred embodiment depicted in FIGS. 1a-1k, a femoral component 25 with guides 500 serving as guiding features 10, oblique pyramids 1605 serving as integrating features 11, and ridges 1100 and oblique pyramids 1605 acting as securing features 12 on the inner surface 2 of the implant 1. The embodiment demonstrates the integration of various features that work in harmony to aid fixation and alignment of the femoral component 25.

The combination of the inner surface 2 and outer surface 3 features of the femoral component 25 is designed to minimise the volume of bone 6 required to be resected.

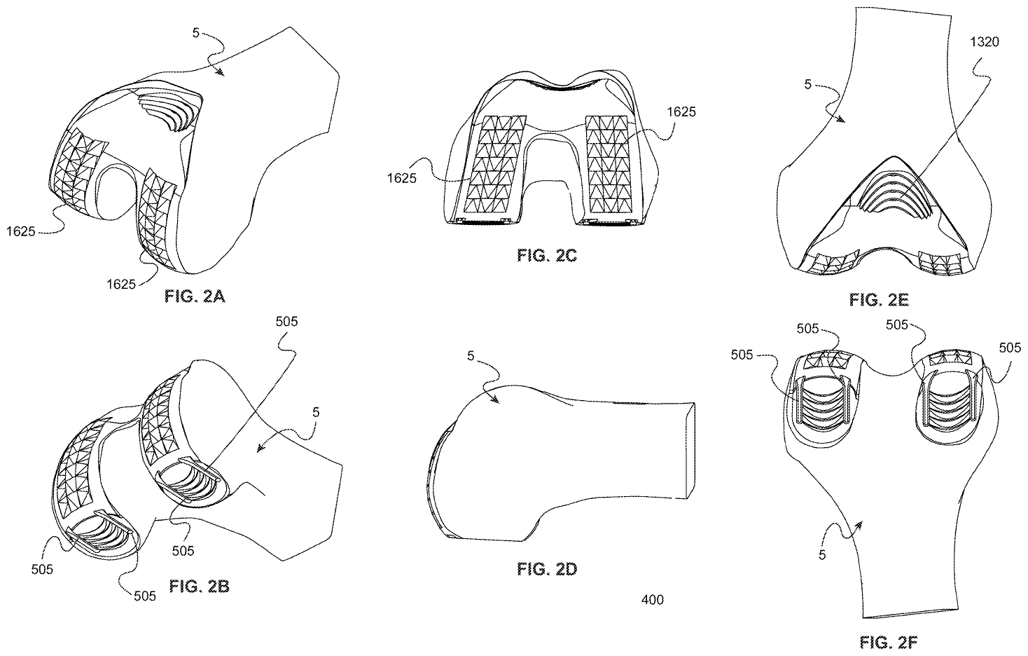

The embodiment of the matching voids 21 required by the femoral component 25 to be resected onto on the femur are depicted in FIGS. 2a-2f. The surface of the bone 6 is shaped to allow the secure seating of the femoral component 25 through the mating of the inner surface geometric features 9 and corresponding matching voids 21. The mating of the inner surface geometric features 9 and bone 6 are dependent on the precise shaping of biological tissue 5.

An implant 1 experiences large static and dynamic forces as it functions. In addition, the amount of biological tissue 5 that is required to be removed, to allow for implantation, should be minimised.

The design and configuration of the geometry is such that it minimises the size and thickness of the implant 1 whilst maintaining the structural integrity of the implant 1 such that it can withstand forces that it encounters in function.

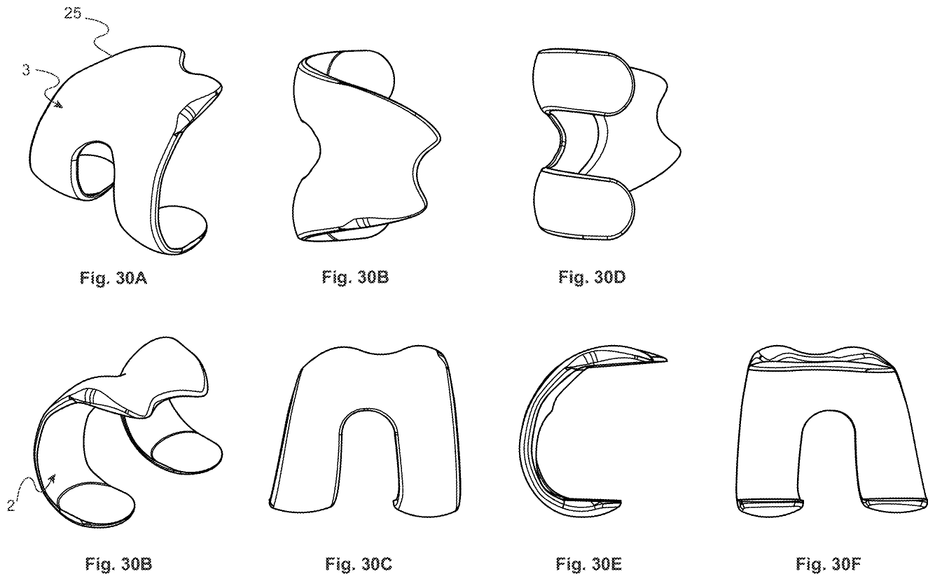

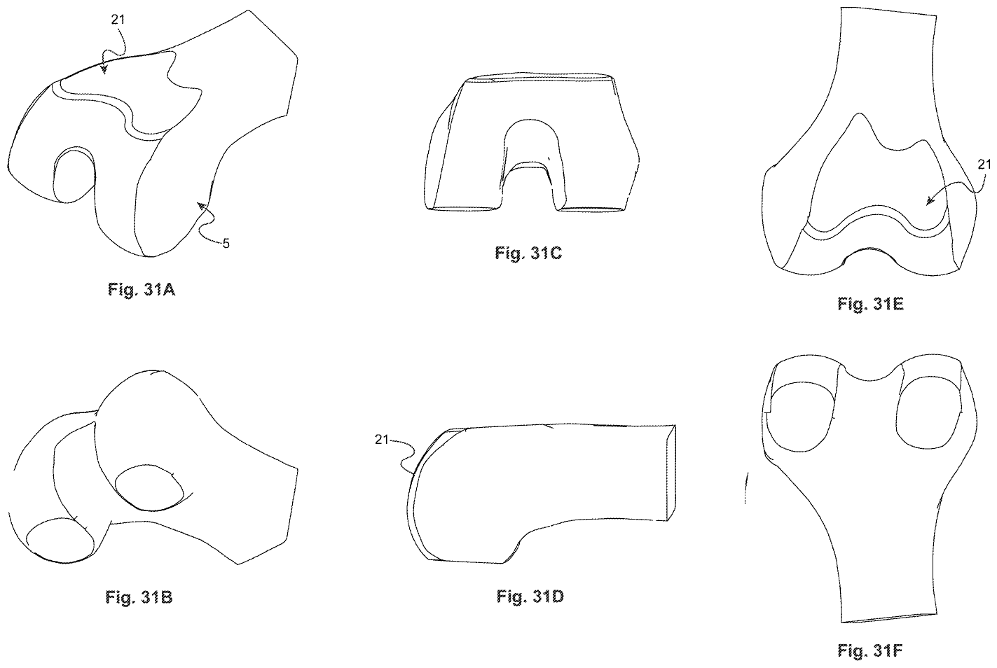

One embodiment, depicted in FIGS. 30a-30f of a femoral component 25, where the inner surface 2 geometry of the femoral component 25 approximately follows the outer surface 3 of the femoral component 25. This “shell” minimises the amount of biological tissue 5 that must be resected. The corresponding embodiment of the matching voids 21 of the femoral component are depicted in FIGS. 31a-31f.

The thickness of this shell and other design aspects of the implant 1 will be chosen based on the properties of the material in which the femoral component 25 is constructed from, and the forces encountered in function of the femoral component 25 such that it maintains structural integrity. The implant 1 may be constructed from biocompatible and/or bioinert materials, example materials may comprise of titanium and/or engineering ceramics.

Implant 1 aim to restore function in some biological structure. In the case of joint implants, that involves restoring joint mobility. In the case of total knee arthroplasty, this involves the use of a femoral component 25 which has an articulating outer surface. A key design requirement is the trochlear groove which prevents the patella from subluxation or dislocation.

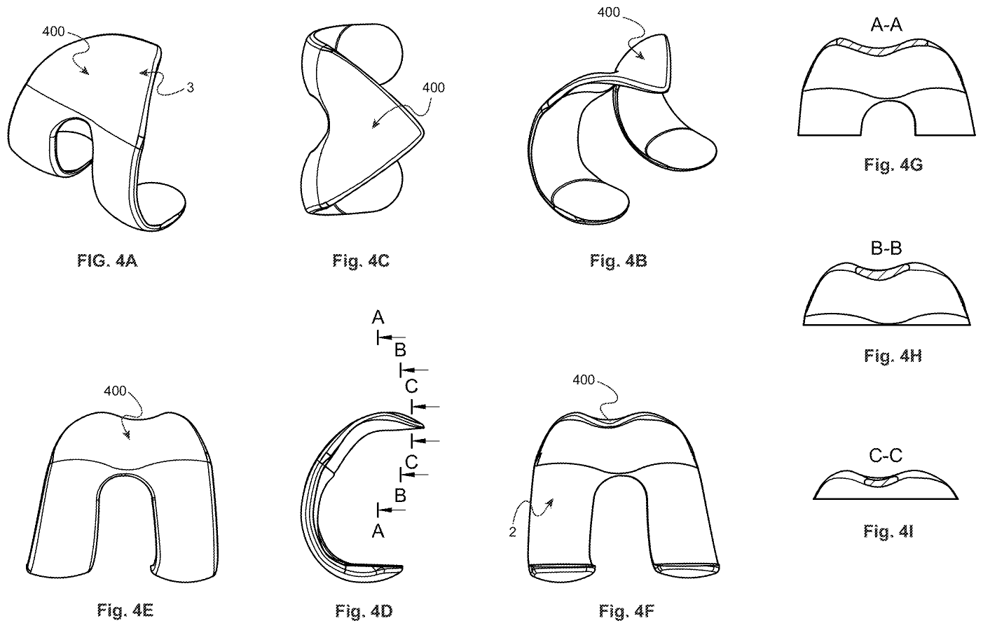

One preferred embodiment of the present invention is a femoral component 25 used in total knee arthroplasties which is depicted in FIGS. 4a-4i. The anterior design of the femoral component 25 minimises the matching resection 20 required to produce the matching voids 21 on the biological tissue 5 while maintaining the required function. An embodiment of the femur with matching voids 21 corresponding to the femoral component 25 is depicted in FIGS. 34a-34f.

The anterior of femoral component 25 is designed such that it follows the functional region of the trochlear groove region 400 on an implant 1. The functional region of the trochlear groove region 400 on an implant 1 is the region where the patella's facets make contact with the femur and keeps the patella tracking correctly, and seeks to replicate and/or replace the function of a natural trochlear groove on the femur in a functional knee in preventing subluxation or dislocation of the patella.

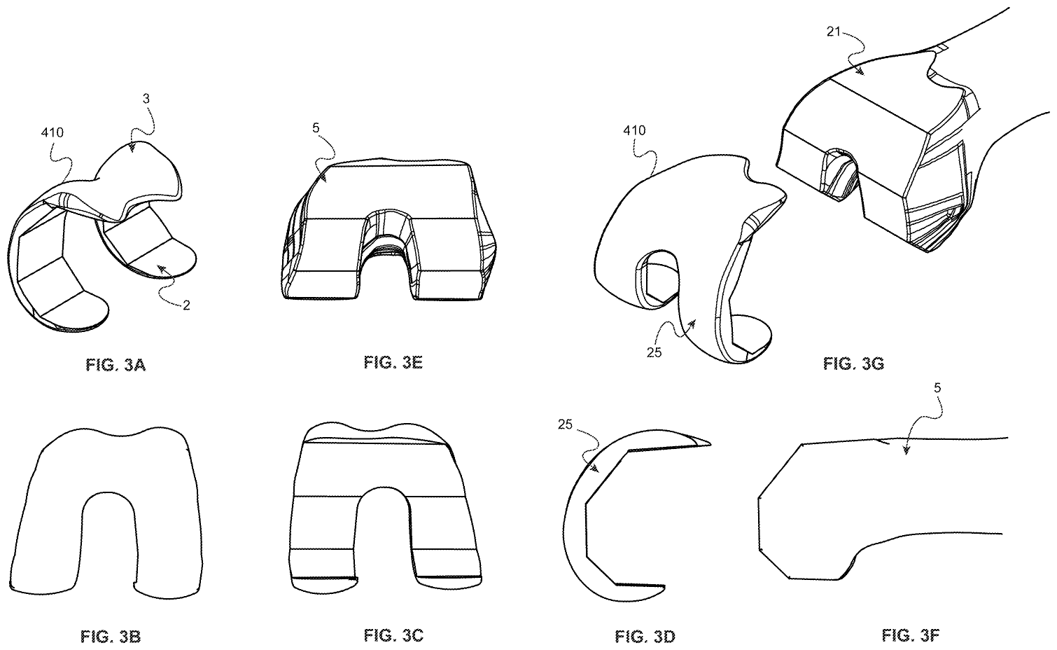

The design allows the trochlear groove region 400 on the biological tissue 5 to be replaced such that it is compatible with the patella or a complementary patella implant (dependent on the design of the outer surface 3 and the surgeon's medical judgement) but minimising removal of the biological tissue 5 from the border between the trochlear groove and the condyle 405. With a conventional implant 410 the border between the trochlear groove and the condyle 405 on the biological tissue 5 is completely removed as depicted in FIGS. 3e-3g.

The final correct seated location of an implant 100 onto, or into, biological tissue 5 is dependent on the insertion direction and orientation.

A guide 500, or series of guides 500, can be favourably employed to control the direction and orientation of the implant as it is being inserted to the ideal seated position. Each guide 500 comprises of inner surface geometric features 9 on the implant 1, with the matching voids 21 on the biological tissue 5.

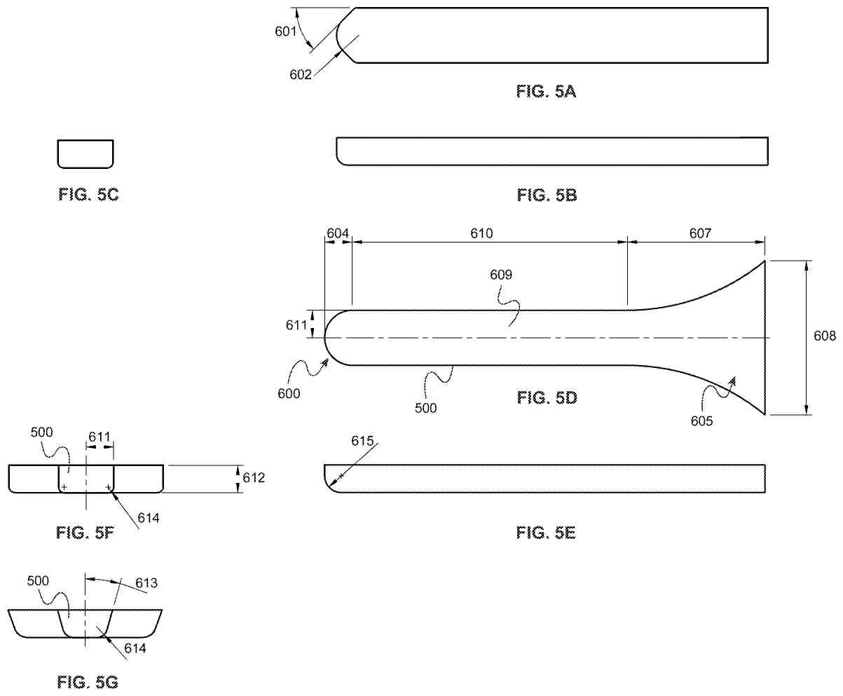

FIGS. 5a-5g illustrates different geometries and dimensions that the guides 500 may be comprised of. A guide 500 may comprise of the tip guide 600, the entry way 605, guide body 609 and round tip radius 603.

The entry way 605 of a guide 600 provides greater tolerance when inserting the implant 1 onto the biological tissue 5. An entry way length 607 and entry way width 608 drives the entry way angle 606 which is the angle of opening from the associated guide 500.

A guide 500 is comprised of the guide body 609 which has a guide length 610. A guide is also comprised of a guide width 611 and guide height 612. A guide body 609 forms a rectangular prism which can create sharp edges which compromise how insertable the implant is. The incorporation of a guide edge fillet radius 614 dulls the guide 500 edges which would engage the biological tissue 5. A guide taper angle 613 and guide edge fillet radius 614 reduces acuteness of guide edges and the biological tissue 5, aiding in the seating of the implant 1 in the desired position.

Guides 500 help guide the implant into it final seated position, however if the tip of the guide 500 cannot easily insert into the matching voids 21, the efficacy of the guides is negatively impacted. The guide tip edge fillet radius 615 creates a continuous surface which better glides into the guides 500.

An embodiment of a series of guides 500 is depicted in FIGS. 1a, 1b, and 1j of a femoral component 25. The guide 500 comprises inner surface geometric features 9 on each of the posterior condyle inner surfaces 2. The guides 500 engage with the matching voids for guides 505 on the biological tissue 5 to assist in seating the implant 1.

Depicted in FIGS. 5a-5g, the tip of the guide 600 is the leading end of the guide 500 when the implant 1 is being inserted into the biological tissue 5. Due to the insertion method and the potential brittleness of the biological tissue 5, there is a potential for the tip of the guide 600 to disrupt the matching voids 21 on the biological tissue 5. If the matching voids 21 are disrupted that may produce inadequate seating of the implant 1 and therefore negatively correlated clinical outcomes. To

The tip of the guide 600 is configured in such a way that it minimised the disruption and damage to the biological tissue 5. This is achieved through geometric features that assist in correcting the insertion pathway and protect the tissue.

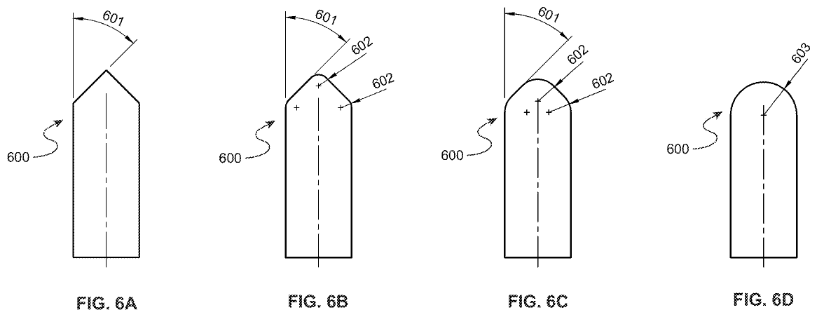

FIGS. 6a-6d depicts multiple embodiments of the feature that may be utilised to mitigate disruption of the biological tissue.

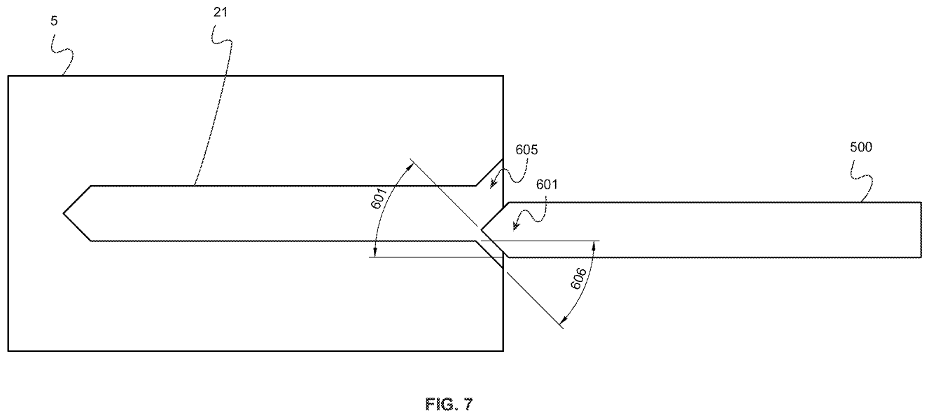

One embodiment of the guiding feature 10 is FIG. 4a, where the profile of the head is triangular. The triangular tip angle 601 may vary between 0 and 90 degrees. This profile is seen in FIG. 7. The matching void 21 on the biological tissue 5 has the entry way 605 such that is angled such that the entry way angle 606 is equal to or less than the triangular tip angle 601. The interaction between the tip of the guide 600 and the entry way 605 assist in correcting the pathway of insertion for the implant by sliding alignment.

Another embodiment of the tip of the guide 600 feature is depicted in FIG. 6b This is similar to the embodiment depicted in FIG. 6a, but the corners of the triangular profile are filleted/rounded/curved on the tip of the guide 600. The triangular tip radius 602 reduces the disruption to the biological tissue 5 when the corners make contact during insertion. The triangular tip radius 602 are commensurate to the size of the guide 600.

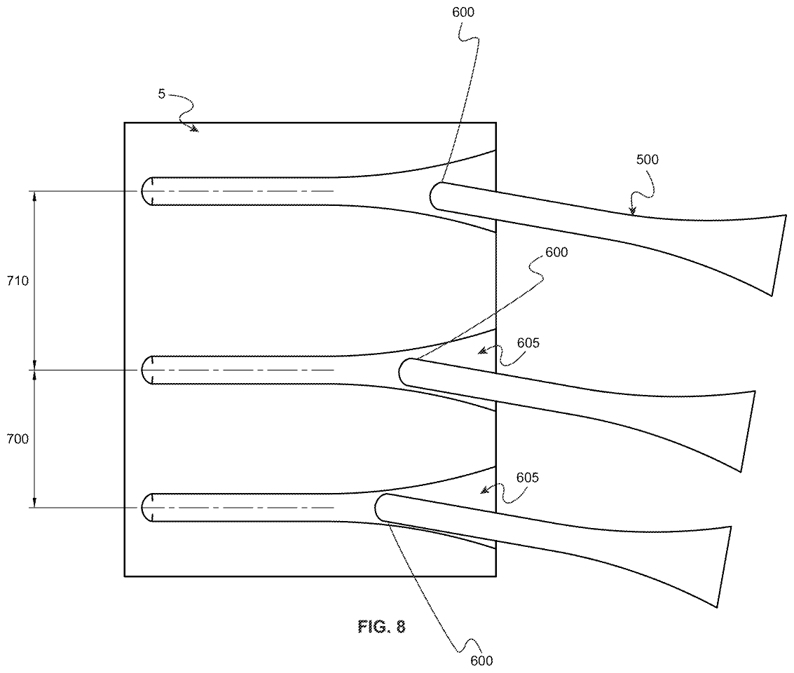

Another embodiment of the tip of guide 600 feature is depicted in FIG. 6d and 8. The tip of the guide 600 is fully rounded. The round tip radius 603 is commensurate to the size of the guide 600. The fully rounded tip greatly minimises the disruption to the biological tissue 5 and provides a pivoting point to assist in correcting the insertion pathway for the guide 600. The entry way 605 for the matching void 21 on the biological tissue 5 is radiused, as depicted in FIG. 8, as well to assist in correcting the pathway of insertion for the implant 1.

The alignment and positioning of an implant can be aided through guiding features 10. With a series of uniformly distributed guides 500, it is possible to guide the implant 1 into and/or onto the matching voids 21 on the biological tissue 5 which do not correspond to the inner surface 2 of the implant 1 resulting in erroneous positioning of the implant 1.

Through non-uniform distribution of inner surface geometric features 9 which act as guiding features 10, it is possible to allow only one means of inserting the implant 1. This mitigates the possibility of fitting the implant 1 incorrectly. This creates asymmetrical inner surface geometric features 9 which allow only one insertable position.

One embodiment of this asymmetry is depicted in FIG. 8. This embodiment demonstrates a series of guides 500 in which the separation between guides 500 is not consistent. For an asymmetrical layout of guiding features 10, the minor guide separation distance 700 and major guide separation distance 710 depicted in the embodiment must not be equal. This removes the possibility of inserting the guides into incorrect matching voids 21.

The increase of inner surface 2 contact area with biological tissue 5 increases the opportunity for osseointegration and the strength of the osseointegration. The use of geometric features that both increases surface area while preserving bone stock is optimal for the fixation of the implant. A geometric feature which is applied to the inner surface 2 of an implant that increases surface area that will interact with biological tissue 5 is essential for reliable cementless implant 1 fixation.

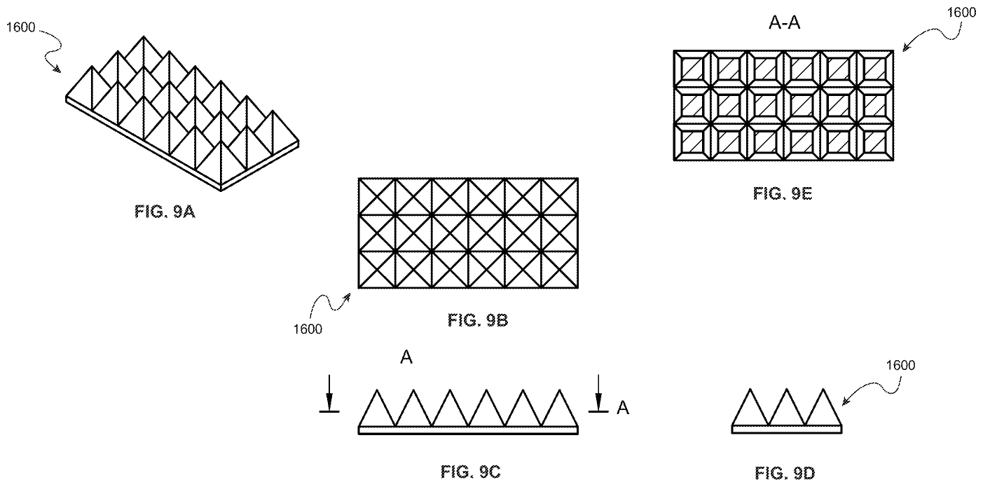

FIGS. 9a-9e depicts an embodiment of pyramid geometry 800 features that may be used as surface-area-increasing geometry, they are pyramid-shaped, consistently distributed and uniform in shape.

The pyramid geometry 800 is one example as an integrating feature 11 which increases the surface area of the inner surface while sparing a significant volume of bone. Through the use of the pyramid geometry 800 depicted, surface area of the internal surface area is appreciably increased compared to an unfeatured surface 812. The percentage increased in surface area due to the configuration of internal surface features may range between 5% to 1000%, this particular embodiment demonstrates an increase of 224% compared to an unfeatured surface. The increase in surface area only results in a minor increase in resection biological tissue 5. This can be mapped onto any implant 1 inner surface to act as an integrating feature 11.

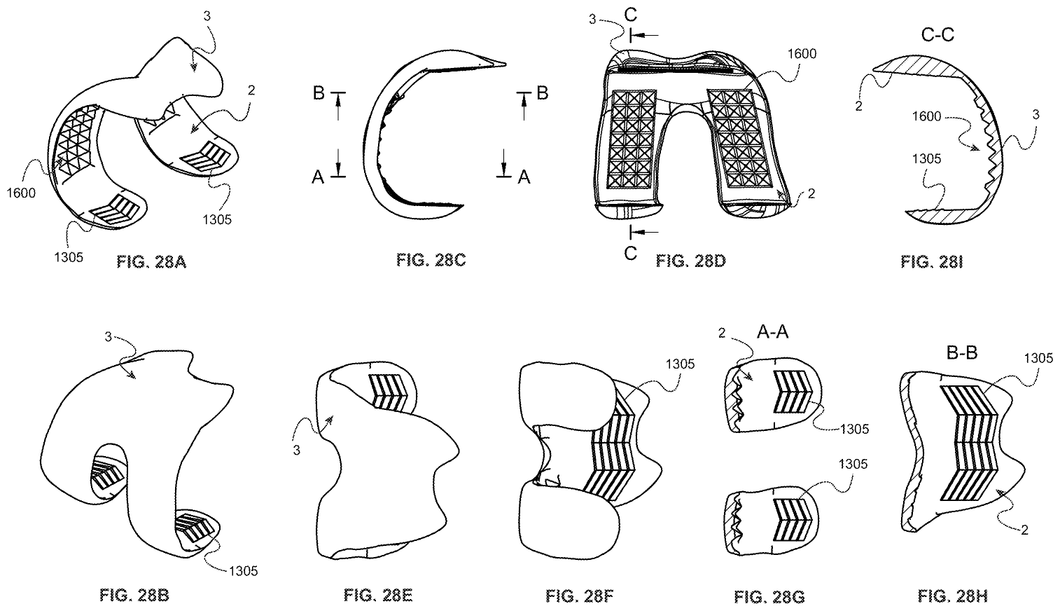

FIGS. 28a, 28d, and 28g depicts an embodiment where the pyramid geometry 800 is mapped onto the inner surface 2 of a femoral component 25 typically used in a Total Knee Arthroplasty (TKA).

FIGS. 50a-50g depicts an embodiment where the pyramid geometry is mapped onto the inner surface 2 of a double uni-compartmental tibial component 30 used in a Total Knee Arthroplasty (TKA).

The pyramid geometry 800 tracks the curved inner surface 2 of the femoral component 25 predominately on the inner distal surface of the implant 1. This ensures the implant is insertable while maximising the region which can act as an integrating feature 11.

FIGS. 41a-41g depict an embodiment where the pyramid geometry 800 is mapped onto the inner surface 2 of a tibial component 30 used in a Total Knee Arthroplasty (TKA). This increases the surface area available significantly for osseointegration with underlying bone 6.

Integrating features 11 encapsulates any geometry which optimises the osseointegration of bone 6 and the implant 1. This geometry can lie on both planar surfaces 810 and irregular surfaces 811.

Ensuring there is minimal space between the implant 1 and biological tissue 5 is critical to the fixation of the implant 1. It is also critical that biological tissue 5 at the interface of the implant 1 is healthy. The biological tissue 5 at the interface of the biological tissue 5 must be viable, otherwise macrophages are required to clear out any dead tissue can also lead to a gap between the implant's inner surface 2 and biological tissue 5.

The use of integrating features 11 are primarily to ensure long-term survivorship of the implant 1. Preparing biological tissue 5 such that it mirrors the geometry of the integrating features 11 will increase contact surface area of the implant 1 while not inflicting significant tissue damage in the process of fitting the implant 1.

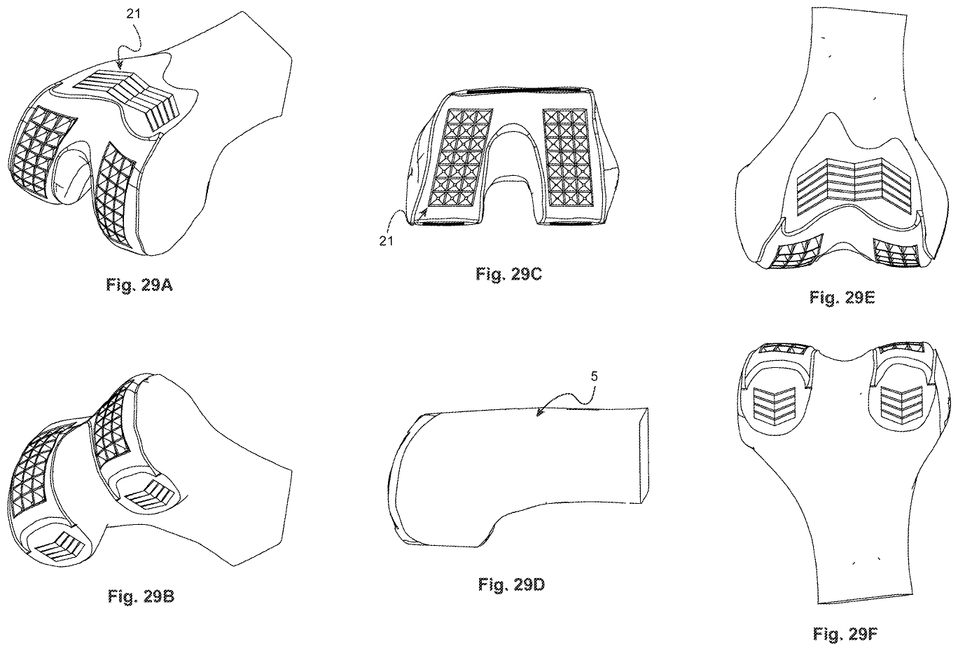

FIGS. 29a-29e of drawings depicts an embodiment of the femur with the biological tissue 5 prepared for the insert of the implant 1 with the matching voids 21 of pyramid geometry 800 integrating features 11. In the embodiment, the femur is the has matching voids 21 corresponding to the inner surface geometric features 9 of the implant 1 to be inserted. The matching voids 21 can be applied to any integrating feature 11 applied to the inner surface 2 of the implant 1.

(Ridges are used to secure the implant to the biological tissue to prevent the implant from coming loose once fitted.)

Securing the implant 1 to biological tissue 5 ensures that the implant remains in the position of insertion until the biological tissue 5 and implant 1 and form a more permanent bond (secondary fixation 50).

FIGS. 3a-3f depicts one embodiment of a conventional implant 410. The embodiment shows a femoral component 25 used in a total knee arthroplasty with matching resections 20 preparing the bone 6. The primary securing mechanism, primary fixation 45, is the friction on the anterior surface 1110 and posterior surface 1111 of the femur.

A ridge 1100, or series of ridges 1100 provide a mechanism to secure the implant 1 to biological tissue 5 once inserted into the ideal seated position. This ensures it does not stray from the position fitted intra-operatively by the surgeon.

Ridges 1100 are positioned orthogonal to axis of insertion 60 and parallel to the ultimate position of the implants 1 inner surface 2. Once the implant 1 is fitted which would require a level a force applied to the implant 1, the ridges 1100 would resist forces acting in the opposite the direction of insertion.

Each ridge 1100 comprises of an inner surface geometric feature 9 on the implant 1, with corresponding matching voids 21 shaped to the final correct seated location of the implant 1 in the biological tissue 5.

Ridges 1100 counteract forces acting to push the implant 1 out of position. There are however practical limitations inserting an implant 1 without causing inflicting damage on biological tissue 5 surface.

A series of ridges 1100 placed on the inner surface 2 of an implant 1 can be used to form a snap-fit like interface. The ridges 1100 are configured in a manner which acts as a securing feature 12 while limiting damage to underlying biological tissue 5.

The biological tissue 5 has a complementary surface preparation such that once the implant is in the final seated position, the ridges 1100 and biological tissue 5 form a locking joint. This joint is referred to hereon as a “substrate deformation joint” 1200, and is based on the reliance of small degrees of deformation in biological tissue 5 to form the joint. This mechanism is dependent on the configuration of parameters associated with each ridge 1100.

One embodiment of a ridge in FIG. 10 depicts a side profile of one configuration a series of ridges 1100 which form a substrate deformation joint 1200. A ridge 1100 can be configured to optimise the performance of the substrate deformation joint 1200. The ridge length 1220 is defined by the peak to peak distance or trough to trough distance in a series of ridges 1100. The ridges are orientated relative to the draft angle 1225 which must be greater than 0° degrees to ensure implantability. The acuteness of the ridges is determined by the joint engagement angle 1226 which increases the strength of the substrate deformation joint as the angle decreases, but also requires precise and accurate matching resections 20. The ridge height 1221 can also increase contact surface area to form a stronger deformation joint 1200, but also requires larger matching resections 20. The tolerance gap 1221 of the ridges 1100 can be configured to compensate for variation in the matching resections 20.

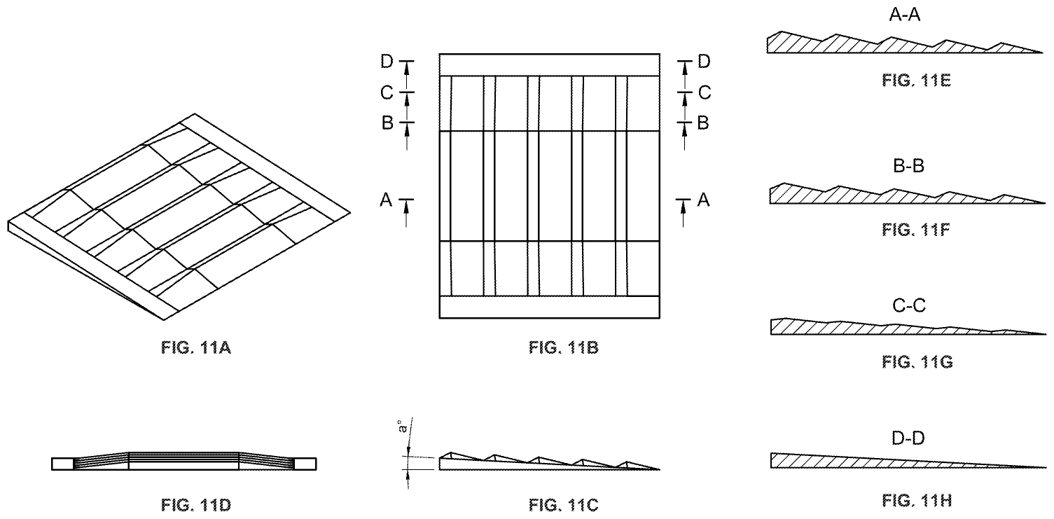

FIGS. 11a-11h depicts an embodiment of a series of ridges 1100 features configured to form a substrate deformation joint 1200.

Depicted in FIG. 11e, a cross-sectional view of a series of the series ridges 1100 is seen. FIG. 10 depicts a technical schematic of those ridges 1100. The series of ridges 1100 lie on the inner surface 2 of an implant 1 and when engaged with biological tissue 5 which is shaped to have matching voids 21 that allow the ridges 1100 to function, form a substrate deformation joint 1200.

The series of ridges 1100 are configured such that they do not fully engage with the biological tissue 5 during the initial insertion, until the series of ridges 1100 engage and deform the biological tissue 5 at the same instance during the final action phase of insertion.

This minimises disruption to biological tissue 5 allow function and securing of the joint. The final action phase of insertion requires the implant to be forcefully pushed onto the biological tissue 5 which will deform, allowing the implant 1 to ride into the ultimate fixation position in the matching voids 21 of the biological tissue 5, forming the substrate deformation joint 1200. In the final position, the substrate deformation joint 1200 resists forces opposite the direction of insertion. In the final position, there is a significant increase in these resistive forces which will aid in the primary fixation 45 of the implant.

FIGS. 44a-44d depicts a representation the final stages of implant 1 insertion from the left side, with the series of ridges 1100 approaching the matching voids 21 and the resulting substrate deformation joint 1200. FIG. 44a-44b depicted the ridges 1100 not engaging, clearing, the biological tissue 5 during the act of insertion as a result of the ridge vertex clearance 1210. The ridges 1100 engage in the second phase of insertion in FIG. 44c-44d forming a substrate deformation joint 1200.

Implants 1 may have forces applied to them either as part of their designed application or incidentally due to their implanted location in the biological tissue 5. Implants 1 which have significant forces applied to them, such as joint implants, could have repeated and multi-directional forces both rotationally and translationally.

Ridges 1100 orientated orthogonal to the axis of insertion 60 do not handle multi-directional forces effectively. Ridges 1100 not uniformly orientated in one direction reduce the likelihood multi-directional forces will lead to the aseptic loosening of an implant 1.

Ridges 1100 not orientated orthogonal to the direction of insertion serve not just as a securing feature 12 but also ensure the implant 1 seats in the desired final position of the implant.

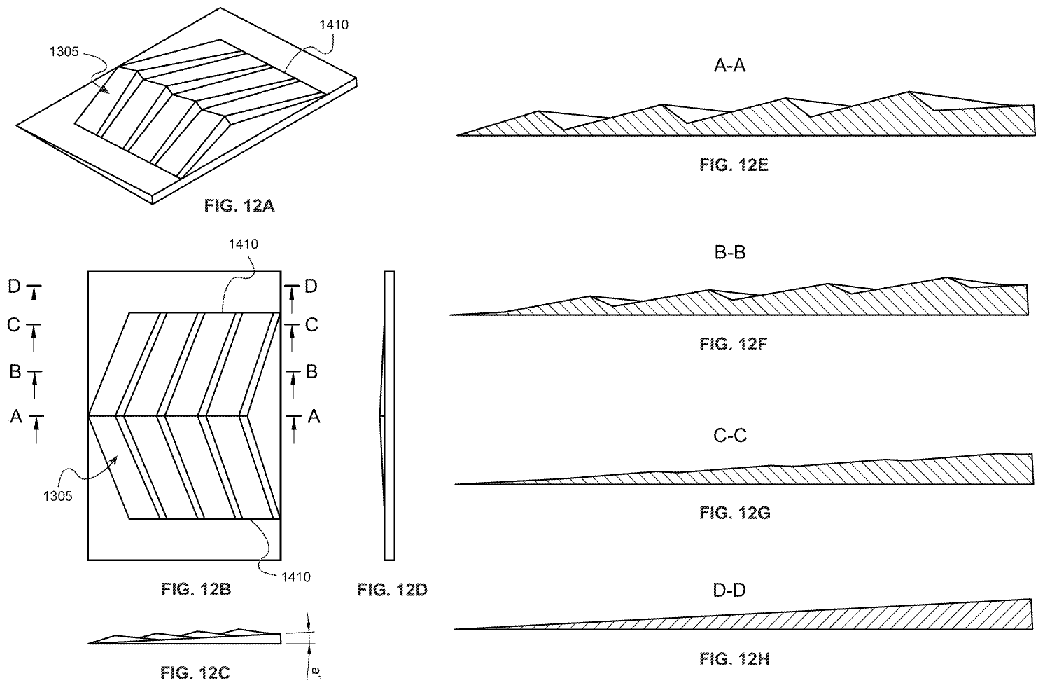

FIG. 12a of drawings depicts an embodiment of the described feature; a series of chevron ridges 1305 which acting as a securing feature 12. The chevron ridge 1305 is an angled ridge feature in which the chevron is angled relative to the angle of insertion. This ensures than an implant 1 with such a feature is still insertable, however the substrate deformation joint 1200 formed is more robust. This embodiment still retains the minimisation of disruption to biological tissue 5 during insertion design of the embodiment depicted in FIGS. 11a-11h due to the similar profile utilised as depicted in FIG. 12e.

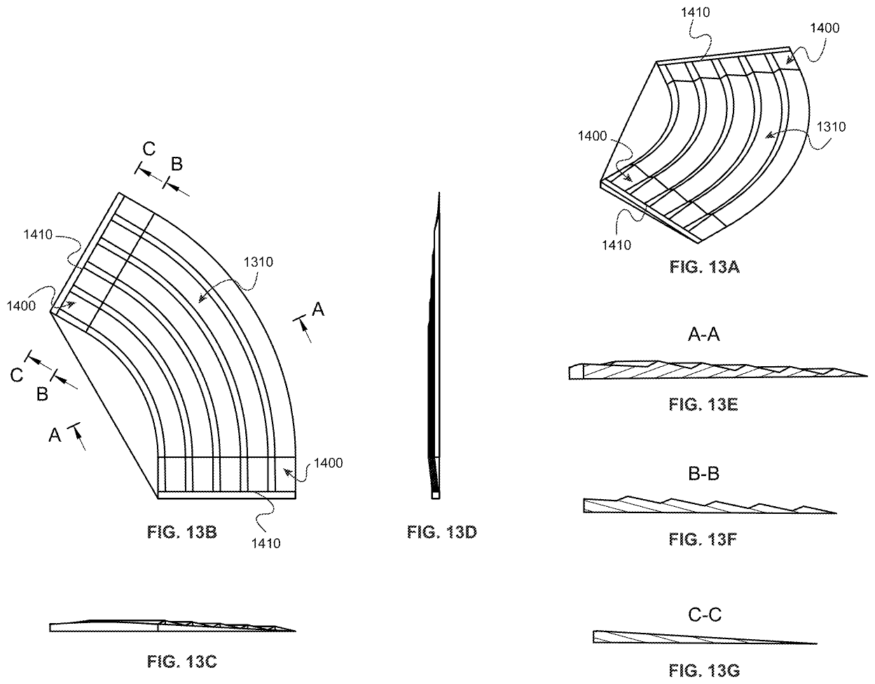

FIGS. 13a-13g depicts an embodiment of a series of curved ridges 1310 features acting as a securing feature 12. The embodiment of curved ridges 1310 demonstrates a ridge feature that does not involve linear geometries. The benefit of curved ridges 1310 are their ability to most efficiently handle multi-directional forces. This embodiment still retains the minimisation of disruption to biological tissue 5 during insertion design of the embodiment depicted in FIG. 11a-11h due to the similar profile utilised as depicted in FIG. 13e.

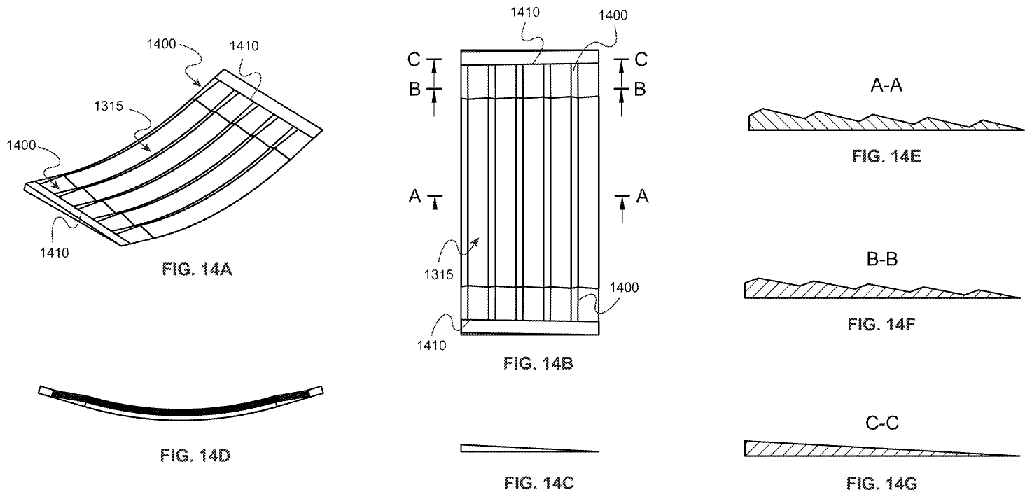

FIGS. 14a-14g depicts an embodiment of a series of rounded ridges 1315 features acting as a securing feature 12. Rounded ridges 1315 are an embodiment of a securing feature in utilising ridges which sit on a non-planar surface. The series of rounded ridges 1315 lie on a curved surface, demonstrating the application of ridges 1100 on an irregular implant inner surface 2. This embodiment still retains the minimisation of disruption to biological tissue 5 during insertion design of the embodiment depicted in FIGS. 11a-11h due to the similar profile utilised as depicted in FIG. 14e.

The preferred embodiment depicted in FIGS. 1a-1j utilises rounded ridges 1315 and curved ridges 1310 combined to form a securing feature 12 on the inner surface 2 of a femoral component 25. The embodiment demonstrates a combination of ridge 1100 features can be amalgamate into one securing feature 12 which assists primary fixation 45 of the implant 1.

The ridges 1100 on the implant 1 and the corresponding matching voids 21 require more precise bone shaping to function compared to other inner surface geometric feature 9. The jamming of the ridges is a potential issue that may occur due medial-lateral inaccuracies associated with insertion of the implant 1, which causes the ridges to disrupt the biological tissue 5 that forms the matching voids 21 such that a substrate deformation joint 1200 is not able to be properly formed.

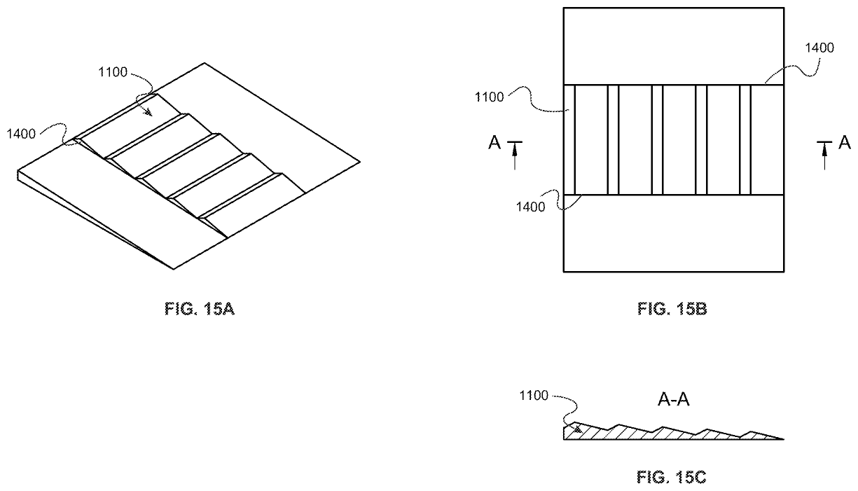

This is particularly apparent on the ridge termination edge 1410 in FIGS. 15a-15c. Due to medial-lateral inaccuracies during implantation, an abrupt termination of ridges on the ridge termination edge 1410 can result in the potential for ridges 1100 to ride up and out of the matching void 21 onto the surface of the biological tissue and disrupt the matching void 21 and the surrounding surface. The disrupted matching voids 21 cannot form a true substrate deformation joint 1200 and removal and reimplantation of the implant would also not form a substrate deformation joint 1200.

The fadeout of ridges 1100 as they approach the ridge termination edge 1410 provides a means of more safely terminating ridges 1100 such that they can form a substrate deformation joint 1200. The ridge fadeout 1400 provides some tolerance for medial-lateral inaccuracies and reduces the risk of jamming or damaging the matching voids 21 before the implant reaches the final seated position.

The fading out of the ridge termination edge 1410 also reduces the amount of stress raisers associated with the feature.

An embodiment of the feature is depicted in FIGS. 12a-12h. Cross-sections of the chevron ridge 1305 features depicted in FIGS. 12e-12h highlight the ridge fadeout 1300 of the ridge 1100 features to the ridge termination edge 1410. The reduction of the severity of the ridge 1100 feature provides greater tolerance when fitting the implant 1 and thus a reduction in the likelihood of jamming and not forming a robust substrate deformation joint 1200.

Another embodiment of the feature of a ridge fadeout 1300 is depicted in FIGS. 13a-13g. FIGS. 13e-13g illustrate a ridge fadeout 1300 to ridge termination edge 1410 of the curved ridge 1310 features with comparable benefits to that depicted in FIGS. 12a-12h.

Another embodiment of the feature depicted of a ridge fadeout 1300 is depicted in FIGS. 14a-14g. FIGS. 14e-14g illustrates a ridge fadeout 1300 to the ridge termination edge 1410 of the rounded ridges 1315. This is particularly useful as it results in a fadeout in two planes resulting in a clean ridge termination edge 1410. This also somewhat serves as a guiding feature 10 with the ridges 1100 guiding the implant 1 into the optimal position seated position.

Ridge fadeout 1300 features provide significant benefits however have some practical limitations. As a ridge fadeout 1300 approaches the ridge termination edge 1410, the ridges 1100 become increasingly fine which poses practical challenges. The contact surface area of ridges becomes increasingly small which reduces the probability of useful interaction and resulting substrate deforming joint 1200. The structural limitations of the underlying biological tissue 5 which when complementary resections of the implant 1 inner surface 2 ridges 1100 are performed would result in sections of biological tissue 5 which would not have the integrity required to form a substrate deformation joint 1200.

The underlying challenge of ridge fadeouts 1400 is the reduction in surface area and resulting loss in integrity of the biological tissue 5. A means of terminating ridges 1100 such fixation strength is maintained across the entire length of the ridge is beneficial. Through combining securing features 12 and guiding features 10, it is possible to get the most effective combination of both ridges 1100 and guides 500.

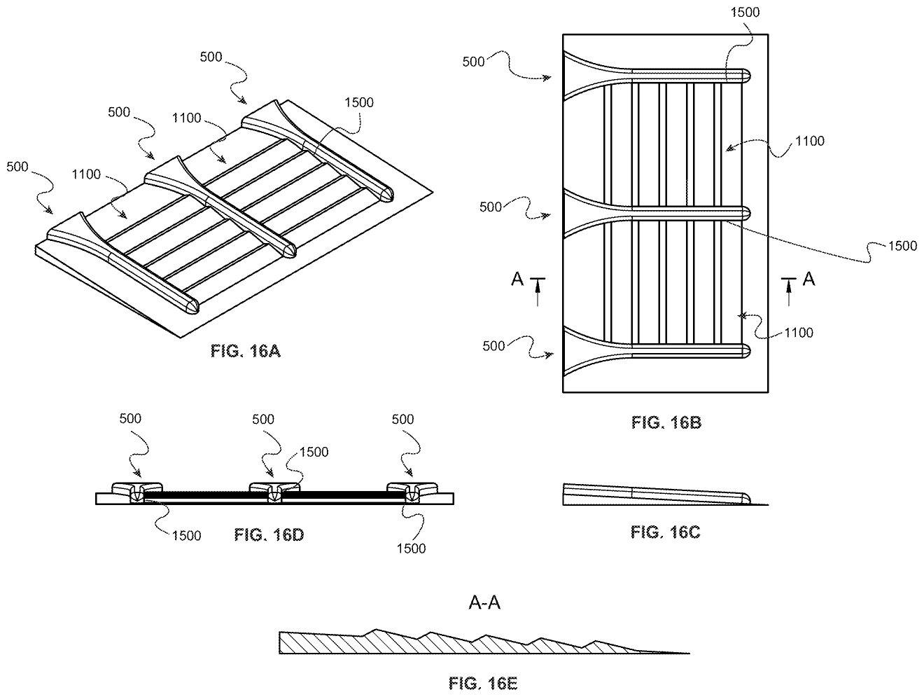

The combination of ridges 1100 and guides 500 which lie on at what would typically be the ridge termination edge 1410 provides a simple solution to ridge termination. In the embodiment FIGS. 16a-16d, ridges 1100 intersect with and are terminated by guides 500, forming a ridge-guide interface 1500.

The benefit derived from the combination of ridges 1100 and guides 500 is the reduction in the regions of ridges 1100 where contact surface area is low. It also provides a means of ensuring ridges 1100 align, which increases the robustness of the substrate deformation joint 1200. This ultimately ensures the final seated position of the implant 1 correlates to the resections performed on the biological tissue 5.

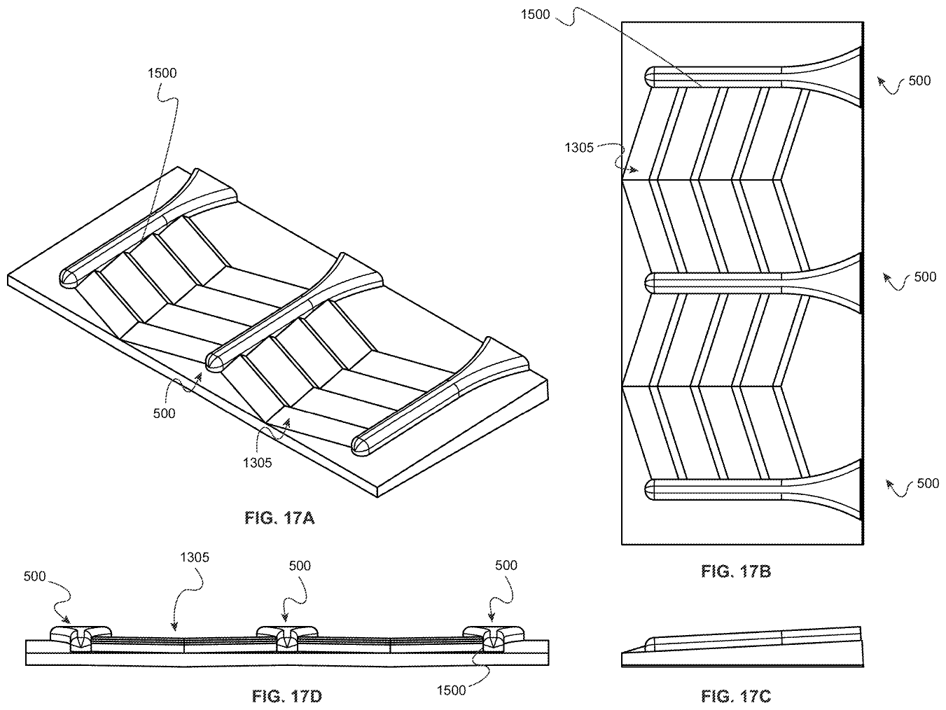

In the embodiment depicted in FIGS. 17a-17d, the use of guides 500 with chevron ridges 1305 can be seen to provide a reliable means of fitting the implant 1. Chevron ridges 1305 are very reliant on alignment and the correct insertion of the implant to form a substrate deformation joint 1200. Through guides the final position is more assured without vulnerable sections of ridges 1100 with a modest surface area.

Primary fixation 45 of the implant 1 is important to the long-term survivorship of the implant 1. Secure primary fixation 45 is positively correlated with secondary fixation 50 and therefore long-term successful clinical outcome.

The interlocking inner surface geometric features 9 mechanically engages with the biological tissue 5, which assists primary fixation 45. The complementary biological tissue 5 is substantially shaped such that it engages with inner surface geometric features 9. The form of the engagement comprises of mechanical interlock, interference or friction fit (jamming).

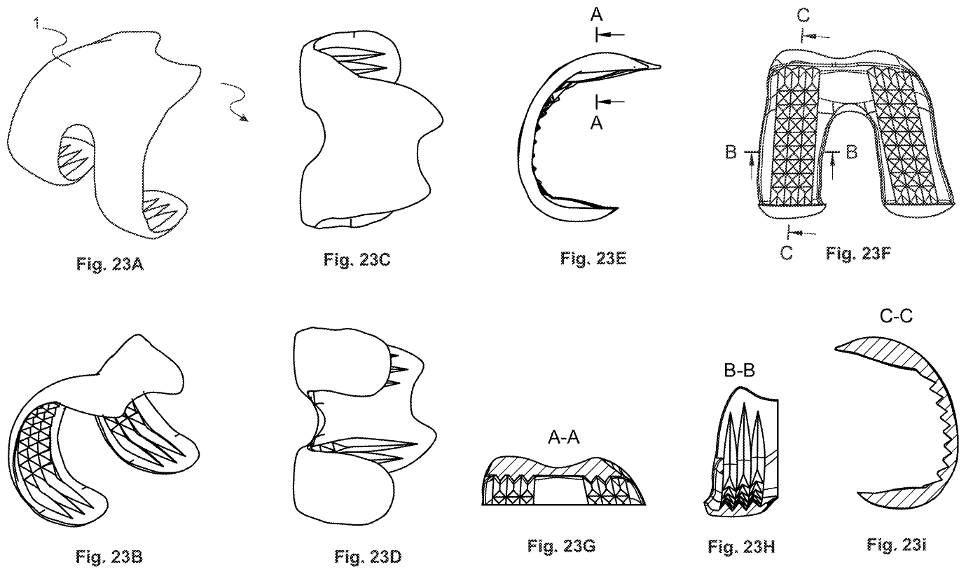

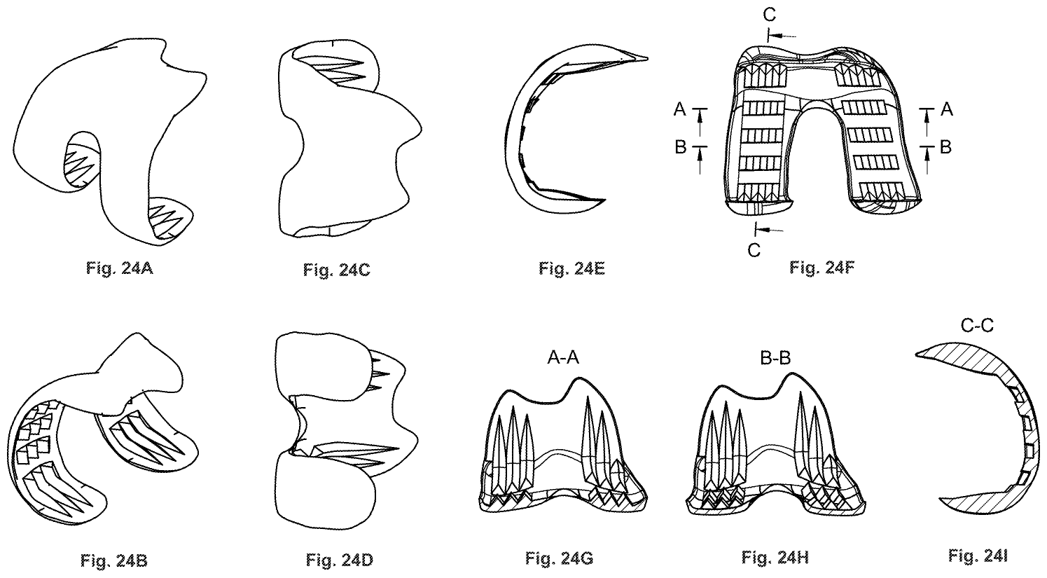

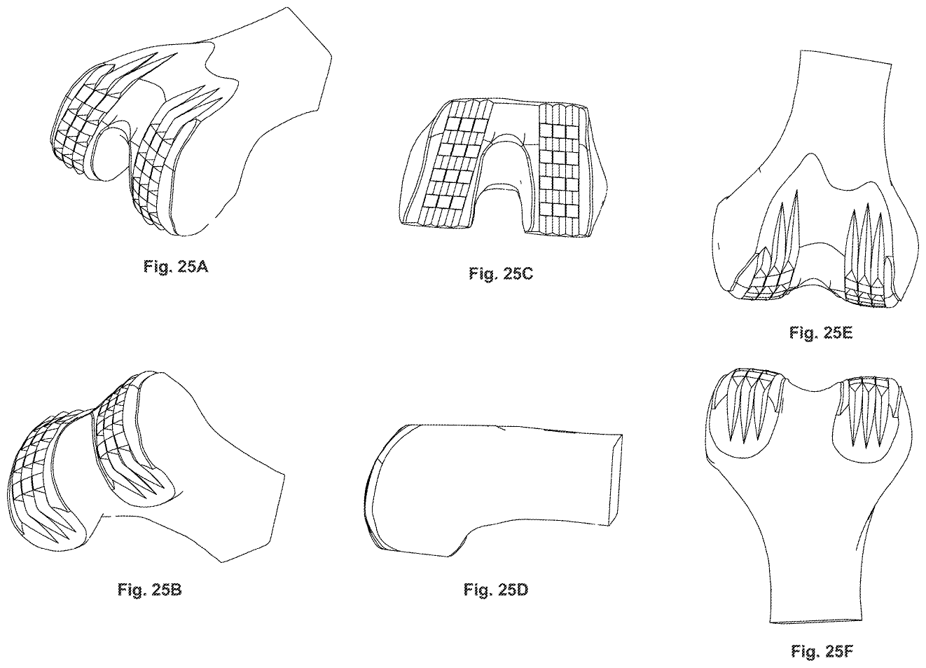

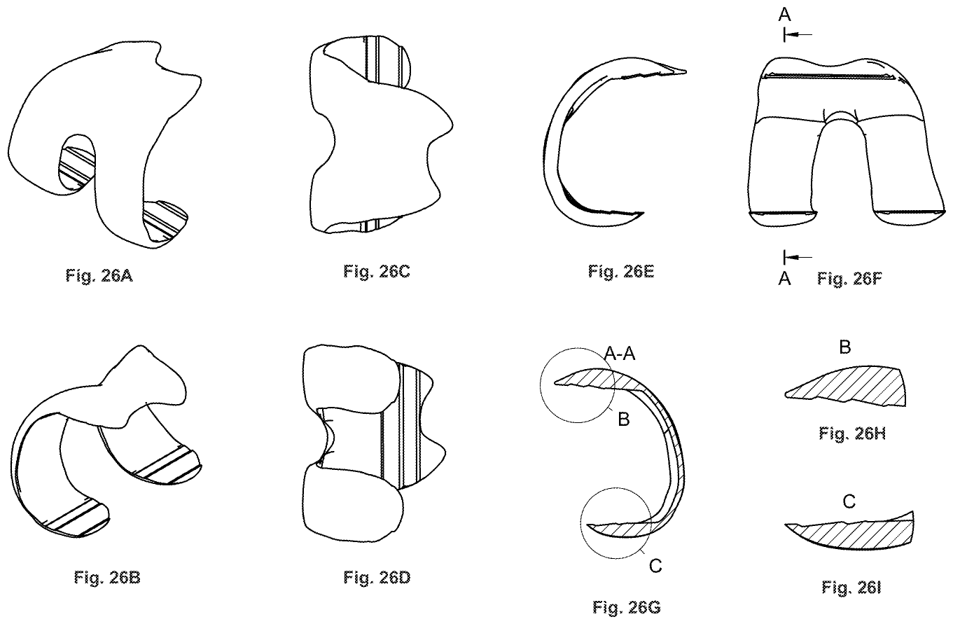

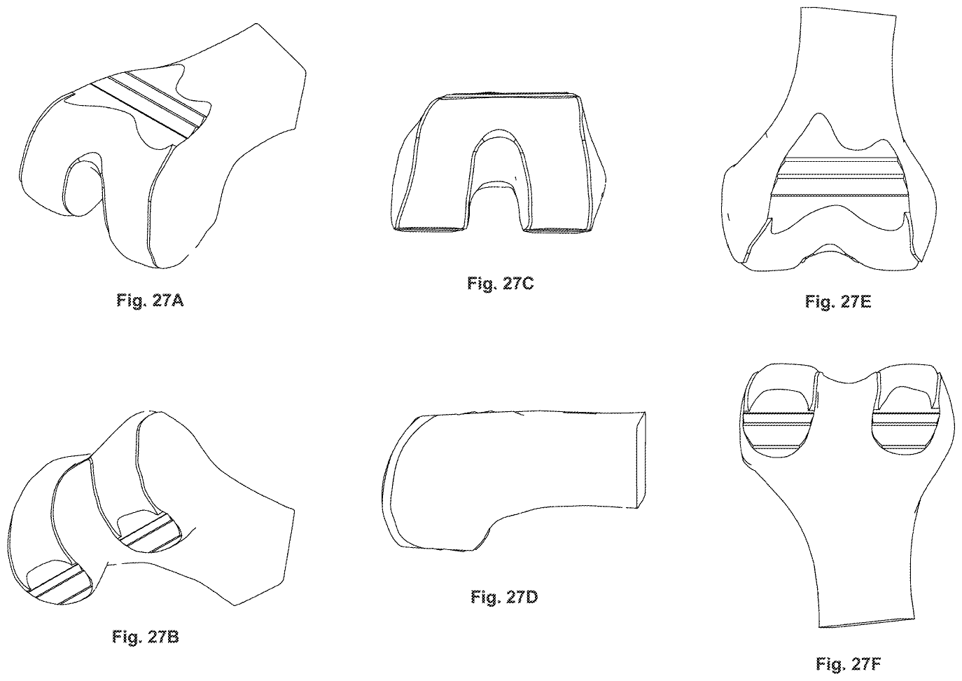

The inner surface geometric features 9 are configured in such a way that they allow the placement of the implant 1 on the biological tissue 5. An embodiment of these feature is that the inner surface geometric features 9 align with the axis of insertion 60 as depicted in FIGS. 23a-23i.

The substantial macroscopic features by the inclusion of the inner surface geometric features 9 increases the likelihood of mechanical engagement and thus the prevention of relative motion and disengagement between the implant 1 and the biological tissue 5. This is correlated with positive clinical outcomes.

One such embodiment of the inner surface geometric features 9 is depicted in FIGS. 9a-9e. They are a uniform pattern of regular pyramids 1600. They are depicted as inner surface geometric features 9 of a femoral component 25 in FIGS. 28a-28i.

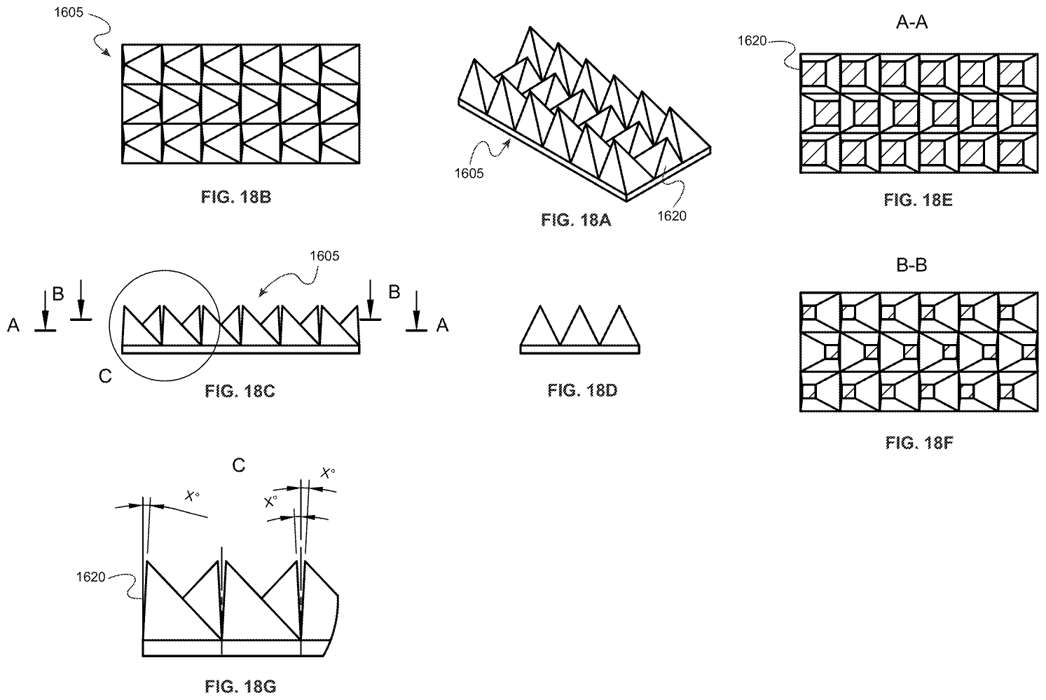

Another embodiment of the inner surface geometric features 9 is depicted in FIGS. 18a-18g, which depicts a pattern of oblique pyramids 1605. The acute angle between the surface of the pyramid and axis of insertion 60 creates an acute angled surface 1620 which allow for greater mechanical engagement with the complementary shaped biological tissue 5. The pattern of features may compromise of features that are not oriented in the same direction. This is depicted in this embodiment where the columns of oblique pyramids 1605 are orientated 180 degrees relative to each other. These differently orientated features are used in concert to produce a beneficial jamming/wedging effect and thus assist mechanical engagement. They are depicted as inner surface 2 features of a TKA femoral component 25 in FIGS. 1a-1e.

An embodiment of a pattern of oblique pyramids 1605 on the inner surface 2 of the tibial component 30 is depicted in FIGS. 38a-38i. The tibial component 30 typically faces forces which result in micromotion of the component, which can be aided by the oblique pyramids 1605 which act as both securing features 12 and integrating features 11.

Another embodiment is depicted in FIGS. 46a-46h and FIGS. 47a-47e where the pattern of oblique pyramids 1605 are used on the inner surface 2 of the patella component 40 and the matching voids 21 are resected in the biological tissue 5, the patella bone. The patella component 40 may be used as a component in a TKA.

Another embodiment is depicted in FIGS. 48a-48h and FIGS. 49a-49f where the pattern of oblique pyramids 1605 are used on the inner surface 2 of the patella component 40 and the matching voids 21 are resected in the biological tissue 5, the patella bone. The patella component 40 may be used as a component in a TKA.

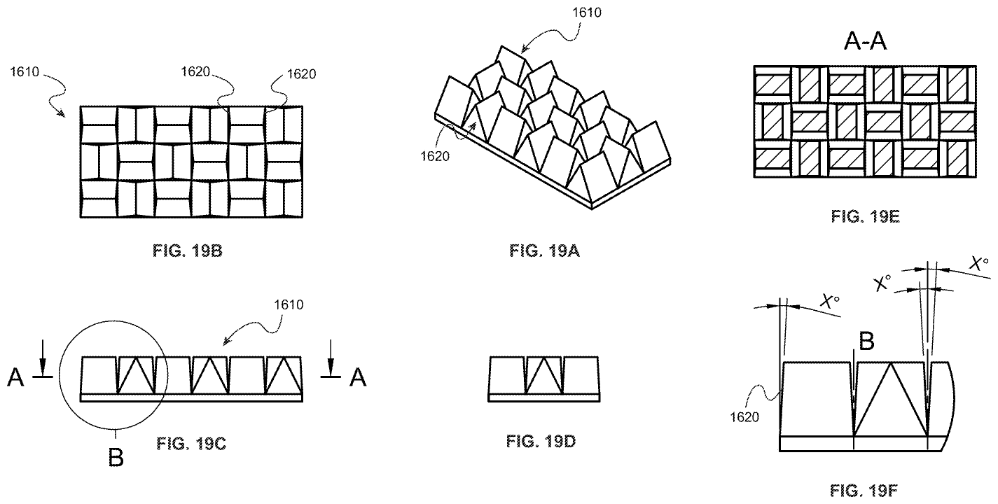

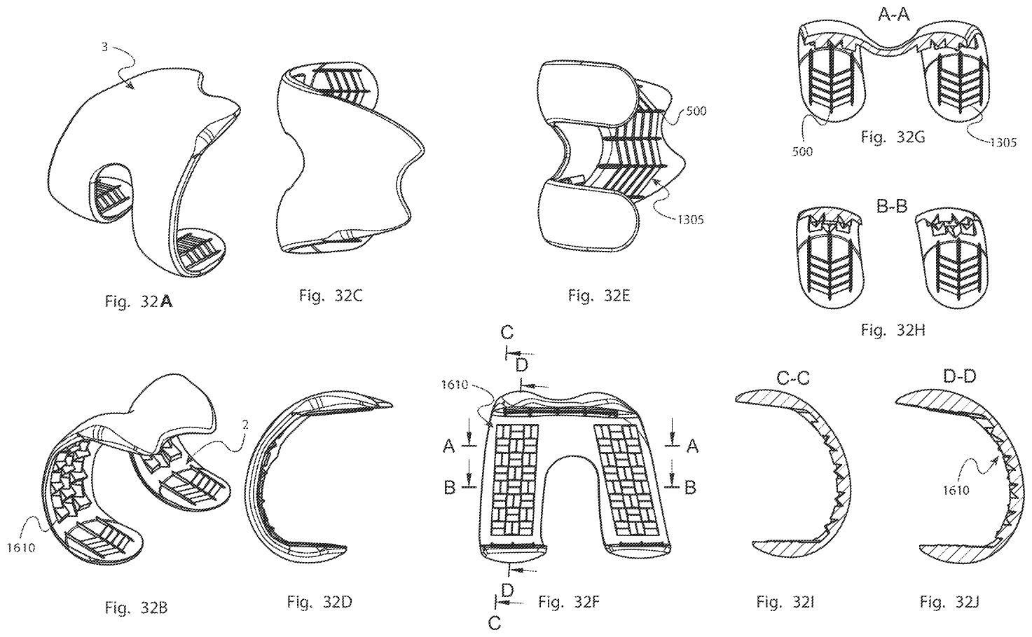

Another embodiment of the inner surface geometric features 9 is depicted in FIGS. 19a-19f. There are uniform wedges 1611 organised into a checkerboard pattern where wedges 1611 rotate 90°. They double the number of acute angled surfaces 1620 compared to the previous embodiment of oblique pyramids 1605. They are depicted as inner surface 2 features of a TKA femoral component 25 in FIGS. 32b-32f.

Another embodiment of the checkerboard pattern of wedges 1611 forming the inner surface 2 of a tibial component 30 is depicted in FIGS. 40a-40i. The inner surface geometric features 9 that assist in primary fixation 45, acting as securing features 12, may also in addition provide increased surface area, acting as an integrating feature 11. As previously mentioned, increased surface area is positively correlated with high quality secondary fixation 50, which comprises of increased speed of osseointegration and/or increase osseointegration strength between the implant 1 and biological tissue 5. Good secondary fixation 50 is positively correlated to good clinical outcomes.

The increase in surface area and proximity to bone due to the jamming effect of inner surface geometric features 9 increases opportunity for osseointegration. This is seen in the embodiment of the oblique pyramids 1605 features depicted in FIGS. 18a-18h, what have also been utilise on the preferred embodiment depicted in FIGS. 1a-1k. The amount of surface area of the inner surface 2 is increased when oblique pyramids 1605 features are utilised compared to if there are no inner surface geometric features 9 and the features assist in primary fixation 45.

Secondary fixation 50 takes place over long periods of time and relative motion between the implant 1 and biological tissue 5 prevents secondary fixation 50 to occur. If the secondary fixation 50 is of low quality, then the stability and long-term survivorship of the implant and therefore clinical outcome, is negatively correlated.

The inner surface 2 features, additionally to assisting in primary fixation 45, assist in the transference of rotational and translational forces from the biological tissue 5 through to the implant 1. This minimises the relative motion (micromotions) between the biological tissue 5 and the implant 1 and thus increases the quality of the secondary fixation 50 as it develops.

One embodiment of these inner surface geometric features 9 is seen in FIG. 18g. The oblique pyramid 1605 which has one of its faces acutely angled from the axis of insertion 60 can, compared to an unfeatured surface 812, more effectively transfer force from biological tissue 5 into the implant 1. The transfer of force using the inner surface 2 features also reduces the apparent force parallel to the bone-implant bond interface, allowing it to grow.

Another embodiment in FIGS. 51a-51g depicts a tibial component 30 which is has a combination of regular pyramids 800 and elongated pyramids 1800 on the inner surface 2 of the component. Regular pyramids 800 aid translation of forces, however elongated pyramids 1800 which sit further into the biological tissue 5 help prevent any relative motion of the implant 1.

Forces and moments acting on a joint and thus the implant 1 are not uniform in magnitude and direction. Inner surface geometric features 9 such as the oblique pyramids 1605 depicted in FIGS. 45a-45f, that are all orientated in the same direction may not be best suited to receive and transfer forces or moments in other directions.

Inner surface geometric features 9 that are oriented in different directions can more fully receive and transfer forces through the implant. This reduces relative motion between the implant 1 and the biological tissue 5.

One embodiment is depicted in FIGS. 18a-18f. Shown are alternating columns of oblique pyramids 1605 as inner surface geometric features 9 where each column is 180 degrees from the previous one. This allows for effective transference of force in both the positive and negative direction of the axis of insertion 60.

One embodiment is depicted in FIGS. 19a-19f. Shown are checkerboard arrangement of wedges 1610 inner surface geometric features 9. Alternating wedges are orientated 90 degrees to each other. This allow the effective transference of force in 4 directions and substantial transference in all directions.

Assessment of the orientation required for the effective placement of an implant 1 is regularly carried out preoperatively. The placement of the implant 1 should maximally restore function, however the accuracy and precision of the orientation obtained from preoperative assessment is limited due various reasons. The ability to resect the biological tissue 5 required for the ideal orientation of an implant 1 is also limited due to various reasons. The combination of these two factors requires the surgeon to intraoperatively perform a “trial” of the implant 1 that they are proposing to utilise to assess whether the implant would be oriented in the optimal position to restore function.

In the current state-of-the-art, conventional implants 410 as depicted in FIGS. 3a-3d, utilise trial implants 2000 that have the same inner surface geometric features 9 with addition features to the primary implant 2005 (the implant that is the permanent implant) that increase the ease of extraction of the trial implant 2000 after trial implantation. Trial implantation occurs after the primary matching resections 20 are completed on the biological tissue 5 that allow the implantation of the primary implant 2005. The trial implant 2000 is seated on the matching void 21, providing the surgeon the opportunity to make an informed assessment (aided and/or unaided by mechanical or computer means). This assessment involves identifying whether the placement of an implant 1 with existing resections would restore of joint function. The surgeon may choose to adjust the orientation based on the assessment and performs any required resections to adjust the primary implant 2005 orientation and alignment.

An implant 1 design that utilises the method of implant design as specified in this patent including, the preferred embodiment depicted in FIGS. 1a-1k and 2a-2f, cannot easily utilise a trial implant 2000 that have the same inner surface geometric features 9 as the primary implant 2005. There is a lack of adjustment available due to congruent nature of the inner surface 2 of the implant to the matching voids 21 required on the biological tissue 5. Any adjustments to the orientation of the implant 1 after the primary matching voids 21 have been resected would necessitate gaps occurring between the primary implant 2005 and the biological tissue 5 which may lead to poor clinical outcomes or the use of bone cement which is undesirable.

The proposed method of designing and configurating trial implants 2000, is where the inner surface geometric features 9 of the trial implant 2000 would require the initial trial voids 2010, on the biological tissue 5 to be a subset of the primary matching voids 21 required for final implantation with the primary implant 2005. This is where there is an appropriate amount of remaining biological tissue 5, distributed appropriately such that primary matching voids 21 may be accomplished and there are minimal gaps when the primary implant 2005 is implanted. The trial implant 2000 will function sufficiently for any assessment of the implant 1 required.

The method may also incorporate design features that allow the design of the trial implant 2000, including the internal surface geometric features 9, to allow upsizing and downsizing of the trial implant 2000 and thus the downsizing and upsizing of the primary implant 2005.

One embodiment of the proposed method of design utilises inner surface geometric features 9 for trial implants 2000 is illustrated in FIGS. 37a-37d. The trial voids 2010 pattern is resected from the biological tissue 5 illustrated in FIG. 37a. The preoperatively assessed orientation for the trial implant 2000 is illustrated, in FIG. 37b by the preoperatively proposed void 2015 pattern. The trial voids 2010 are a subset of the preoperatively proposed void 2015 pattern.

After assessment, a decision will be made by the surgeon on the implantation, that may comprise of changing the size of the primary implant 2005 and/or adjusting the orientation of the primary implant 2005. Adjusting the orientation of the primary implant 2005 may comprise of adjustments; adjusted change in x 2021, adjusted change in y 2022 and/or adjusted change in angle 2023 as depicted in FIG. 37c. The adjusted proposed voids 2020 pattern is such that it fully contains the trial void 2010 pattern. The matching resection 20 is then executed to produce the required matching voids 21 as depicted in FIG. 37D and the primary implant 2005 may be implanted where the inner surface geometric features 9 fully engage and any gap is minimised.

A method design of trial implants 2000 that achieve the previously stated intention are where the inner surface geometric features 9 of the trial implant 2000 are truncated and/or offset of those in a of the primary implant 2005. This allows the inner surface geometric features 9 of the trial implant 2000 to require the trial voids 2010 on the biological tissue 5 to be a subset of the proposed voids 2020 required for final implantation with the primary implant 2005.

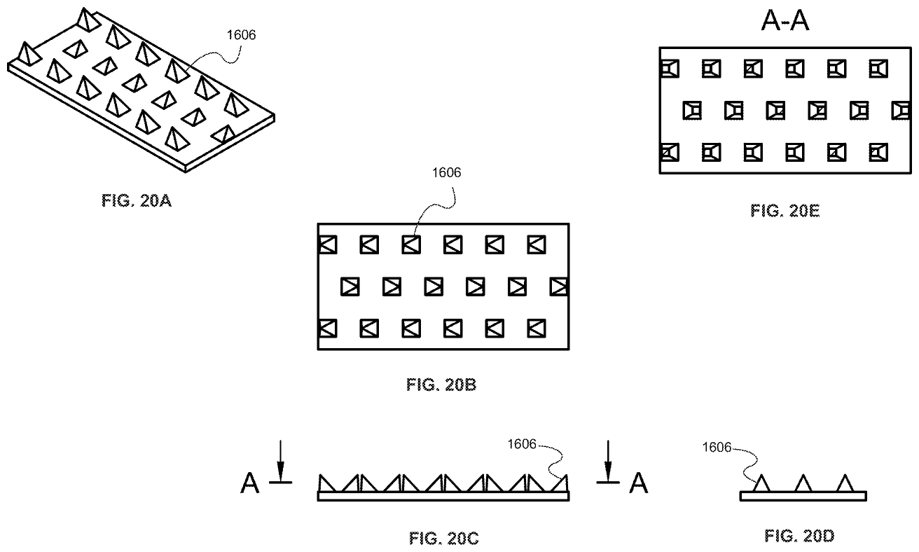

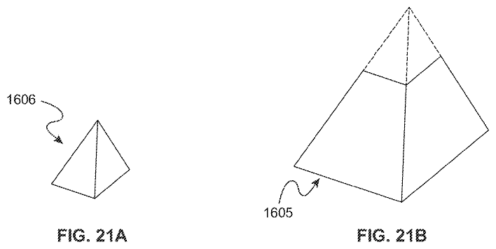

One embodiment of the method of design that uses truncated and/or offset inner surface geometric features 9 are the use of truncated oblique pyramids 1606, as depicted in FIGS. 20a-20d. A truncated oblique pyramid 1606 is the truncated version of an oblique pyramid 1605 as depicted in FIGS. 21a-21b figure. This embodiment demonstrates the feasibility of truncated oblique pyramids 1606 and the opportunities for correction after the fitting of the trial implant 2000.

An embodiment of the use of truncated oblique pyramids 1606 is depicted in FIGS. 36a-36f. The oblique pyramids 1605 features have been truncated and offset on to the inner surface 2 of the trial implant 2000 producing truncated oblique pyramids 1606. The truncated oblique pyramids 1606 features provide similar functionality to the full oblique pyramids 1605 features in transferring translational and rotational forces a trial implant 2000 would experience whilst undergoing assessment. The trial voids 2010 in the biological tissue 5 required for the trial implant 2000 is only a subset of the matching voids 21 required for the full oblique pyramids 1605 other inner surface geometric features 9 for the primary implantation of the preferred embodiment depicted in FIGS. 1a-1k and 2a-2f. This allows for adjustment of the required matching voids 20 for primary implantation whilst retaining full functionality, if it is deemed necessary by the trial assessment.

Preoperatively an assessment of the size of the implant 1 required is done by the surgeon. This sizing may or may not be appropriate for implantation and restoring the function of the organ for various reasons. Intraoperatively there may be a decision to use a smaller or larger sized implant 1 by the surgeon. If the matching resections 20 have already been performed on the biological tissue 5, it may be difficult to utilise an alternate implant 1 size.

The implants 1 may be designed such that different sized implants 1 utilise the same inner surface geometric features 9, or a subset of the same inner surface geometric features 9. The inner surface geometric features 9 are of the same geometry, both in size and in their relative position, while the rest of the implant 1, including the outer surface 3, may be of a different configuration e.g. size. These inner surface geometric features 9 would require minimal additional resections on the matching voids 21 of the biological tissue 5 already resected to allow for implantation and restore function to the organ.

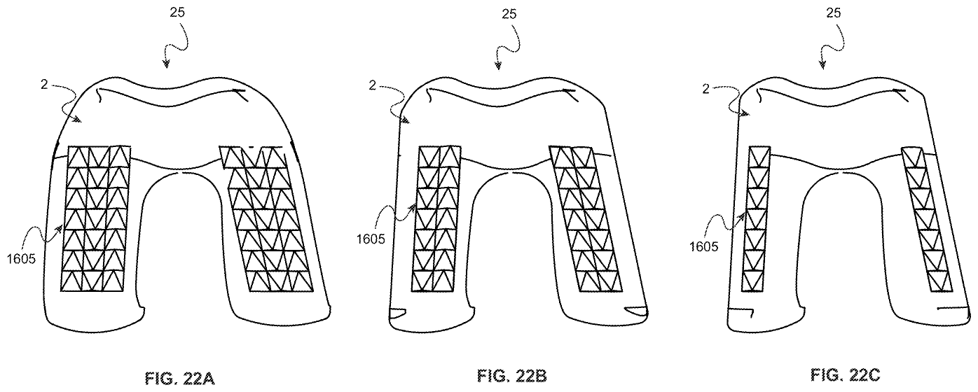

In one embodiment, femoral components 25 are depicted in FIGS. 22a-22c is shown to have same fundamental inner surface geometric features 9, oblique pyramids 1605, over a range of implant 1 sizes. The downsized femoral component 25, FIG. 22c, compared to the femoral component 25, FIG. 22b, has a subset of the inner surface geometric features 9 in the same relative orientation, subset oblique pyramids 1605, which allows them to be used in place of the original implant 1 if it were assessed that a smaller femoral component 25 was required. This is also similar for the upsized femoral component 25, FIG. 22a, with regard to the femoral component 25, FIG. 22b.

Revision surgery is often a consequence where the primary surgery has not achieved its desired clinical outcome. The primary conventional implant 1 must be removed and additional biological tissue 5 is removed before a revision implant able to be implanted.

The primary implant 2005 may be designed and configured in such a way that preserves biological tissue 5, enabling the use of a primary conventional implant 410 for revision surgery.

The primary implant 2005 may be designed in such a way that the matching voids 21 on the biological tissue 5 are a subset of the matching voids 21 required for the implantation of a conventional implant 410. There is enough remaining biological tissue 5, distributed appropriately, that a conventional implant 410 may be implanted. This is considered “bone sparing”, reducing the volume of biological tissue 5 resected for the purpose of seating of the implant 1.

One embodiment that uses this method for a femoral component 25 is depicted in FIGS. 43a-43j. The inner surface geometric features 9 of the femoral component 25 are fully contained within the conventional implants inner surface geometric features 9. The primary matching voids 21 on the biological tissue 5 are a subset of the matching revision voids 2405 that would be required of a conventional implant 410. This results in a spared biological tissue 2400 which is resected in the case of a revision surgery.

This allows for the removal of the primary implant 2005 and reshaping of biological tissue 5, including removal of the spared biological tissue 2400, creating matching revision voids 2405, as depicted in FIGS. 43e-43h, to allow the implantation of a conventional implant 410 as the implant 1 for revision surgery FIGS. 43i and 43j.

Another embodiment is depicted in FIGS. 52a-52c, for a tibial component 30 and 35 spacer. The inner surface geometric features 9 of the tibial component 30, which may be considered the primary implant 2005, are fully contained within the conventional implant's 410 inner surface geometric features 9. The primary matching voids 21 on the biological tissue 5 are a subset of the matching revision voids 2405 that would be required of a conventional implant 410. This results in a spared biological tissue 2400 which is resected in the case of a revision surgery.

The present invention also relates broadly to a surgical orthopaedic implant system for use in a human or animal body. This system preferably utilises the orthopaedic implant of the present invention, and preferably requires a surgeon to undertake certain steps to carry out implantation of the orthopaedic implant. The equipment required in accordance with the surgical orthopaedic implant system of the present invention includes an orthopaedic implant in accordance with the present invention; tools for incising, moving and securing biological tissues (particularly bone, but also tendons, ligaments etc.) encountered in the implantation, tools for precise and accurate shaping of biological tissue (particularly bone) to form matching voids that allow for the function of the features configured on the orthopaedic implant.

The surgical orthopaedic implant system and implantation process comprises the steps summarised in the flowchart depicted in FIG. 53 and further expanded upon in the text below.

Preoperative planning is done to assess various parameters for the implantation process, comprising: the size of the implant; the seated orientation (positioning) of the implant, and the method of approach for accessing the area of interest. This may be done through various methods, typically utilising radiographs.

Intraoperatively, utilising appropriate tools, incisions are made to gain access to the area of interest where the implant is to be seated.

Tissues (ligaments, tendons, etc) in the area of interest are removed or moved and secured, using appropriate tools, to provide access to the area of interest, In addition to providing access they are also moved to so they are minimally effected by the shaping of the biological tissue for implantation, and the risk of inadvertent damage to the tissue is mitigated.

The trial stage of the implantation involves the seating of a trial implant that is utilised to assess and adjust the parameters determined during preoperative planning and the expect result of the procedure.

The trial stage begins with shaping of the biological tissue, in which the implant will be implanted onto, with matching voids. These matching voids orientate the trial implant on the biological tissue as per the determined parameters and also allow for engagement of the biological tissue and the features on the trial implant. Features on the trial implant assist in the assessment of the trial implant, including internal surface features. The features engage with the shaped biological tissue and the matching voids to assist in guiding and securing the trial implant into position. The tools utilised for the shaping of the biological tissue will be capable of accurate and precise shaping to produce the complex matching voids required for the corresponding features on the trial implant.

After the biological tissue is shaped, the trial implant is seated onto the biological tissue. During the process, guiding features on the implant engage with the matching voids on the biological to guide the implant into position. An assessment of the trial implant is performed. The assessment may include motion of the biological tissue, during such motion the securing features on the trial implant engage with the shaped biological tissue and allows no appreciable relative motion between the trial implant and the biological tissue, and allows the assessment to be completed. The assessment determines adjustments, if any, that will be made to the parameters determined during the preoperative planning. These adjustments are chosen to lead to better clinical outcomes. These adjustments may include changing the size of the implant or adjusting the orientation of the planned matching voids for the implant. The trial stage ends with the unseating and removal of trial implant.

Shaping of the biological tissue with matching voids for the implant is then performed. These matching voids orientate the implant on the biological tissue as per the determined parameters and also allow for engagement of the biological tissue and the features on the implant. Features on the implant, including internal surface features, engage with the shaped biological tissue and the matching voids to assist in guiding, securing and integrating the implant into position as determined by the adjusted parameters. The tools utilised for the shaping of the biological tissue will be capable of accurate and precise shaping to produce the complex matching voids required for the corresponding feature on the implant.

The guiding features assist in guiding the implant into the correct orientation. The securing features assist in primary fixation, the initial mechanical attachment of implant to the biological tissue. The integrating features assist in secondary fixation, the process where the body integrates with the implant e.g. osseointegration for bone. These features are further detailed later in the in the text, in the description of the implant.

After the biological tissue is shaped the implant is seated onto the biological tissue. During this process guiding features on the implant engage with the matching voids on the biological to guide the implant into position. The securing features on the implant engage with the shaped biological tissue form mechanical engagement between the implant and biological tissue.

After the implant is seated onto the biological tissue in the correct orientation, the tissue that was previous moved and secure is restored to its native position. The body is then closed up with appropriate surgical techniques.

The integration features are that are engaged with the biological tissue assist in the secondary fixation, e.g. osseointegration, of the implant to the biological tissue over time.

As the present invention may be embodied in several forms without departing from the essential characteristics of the invention, it should be understood that the above described embodiments should not be considered to limit the present invention but rather should be construed broadly. Various modifications, improvements and equivalent arrangements will be readily apparent to those skilled in the art, and are intended to be included within the spirit and scope of the invention. The present embodiments are, therefore, to be considered in all respects as illustrative and not restrictive.

The present invention can be applied to surgical procedures which do not have an embodiment within this patent. The present invention can be extrapolated to arthroplasty and trauma procedures which require the implantation of an implant. A non-exhaustive list of implants in which the present invention applies to includes shoulder hip, finger, ankle and toe implants. The process of implantation would be consistent with the process outlined in FIG. 53, however there may be subtle intra-operative discrepancies inherent with the subtleties of different procedures.

1. An orthopaedic implant for use with or as part of an orthopaedic implant system, comprising:
a body having a bone facing surface configured to mate with a prepared surface of a bone; and
bone engaging means extending from said bone facing surface and being adapted to extend into corresponding cavities formed in the prepared surface of the bone when said body is mated with the bone, said bone engaging means comprising one or more of:
guiding means adapted to guide the implant into its fixated position with the bone;
integration means adapted to promote integration of the implant with the bone; and
securing means adapted to secure the implant to the prepared surface of the bone.

2. The orthopaedic implant of claim 1, wherein the guiding means comprises one or more guide projections extending from the bone facing surface, said guide projections being adapted to extend into corresponding guide cavities formed in the prepared surface of the bone.

3. The orthopaedic implant of claim 2, wherein said one or more guide projections has a substantially uniform width as it extends outwardly from said bone facing surface.

4. The orthopaedic implant of claim 2, wherein said one or more guide projections has a leading portion adapted to promote insertion of the guide projections into the corresponding guide cavities.

5. The orthopaedic implant of claim 4, wherein said leading portion is tapered.

6. The orthopaedic implant of claim 1, wherein said integration means comprises one or more integration projections extending from the bone facing surface, said integration projections being adapted to extend into corresponding integration cavities formed in the prepared surface of the bone.

7. The orthopaedic implant of claim 6, wherein said one or more integration projections tapers as it extends outwardly from said bone facing surface.

8. The orthopaedic implant of claim 6, wherein the one or more integration projections are three dimensional geometric shapes.

9. The orthopaedic implant of claim 6, wherein a shape of the one or more integration projections is adapted to maximise contact surface area of an interface between the implant and the prepared surface of the bone.

10. The orthopaedic implant of claim 6, wherein the one or more integration projections are shaped as a square or rectangular pyramid.

11. The orthopaedic implant of claim 6, wherein the one or more integration projections are adapted to resist transverse movement of the implant relative to the prepared surface of the bone when said body is mated with the bone.

12. The orthopaedic implant of claim 1, wherein said securing means comprises one or more securing projections extending from the bone facing surface, said securing projections being adapted to extend into corresponding securing cavities formed in the prepared surface of the bone.

13. The orthopaedic implant according to claim 12, wherein said one or more securing projections are ridges positioned orthogonal to an axis of insertion of the implant and/or parallel to the bone facing surface once said body is mated with the bone.

14. The orthopaedic implant according to claim 13, wherein said ridges are adapted to form a substrate deforming joint with the prepared surface of the bone, said substrate deforming joint resulting from a resilient deformation of the bone.

15. The orthopaedic implant according to claim 14, wherein said substrate deforming joint is formed when said body is mated with the bone.

16. The orthopaedic implant of claim 14, wherein geometry of said substrate deforming joint biases the implant onto the prepared surface of the bone.

17. The orthopaedic implant of claim 13, wherein the projection of the ridges from the bone facing surface diminishes nearer lateral ends of the implant.

18. The orthopaedic implant of claim 1, wherein the securing means are positioned adjacent to or intersecting with the guiding means.

19. The orthopaedic implant of claim 1, wherein the bone engaging means is adapted to resist rotational and/or translational forces on the implant once said body is mated with the bone.

20. The orthopaedic implant of claim 1, wherein the bone facing surface and/or bone engaging means increases a surface area of an interface between the implant and the prepared surface of the bone by up to 20% relative to an orthopaedic implant with only planar bone facing surfaces.

21. The orthopaedic implant of claim 1, wherein the bone facing surface and/or bone engaging means increases a surface area of an interface between the implant and the prepared surface of the bone by up to 50% relative to an orthopaedic implant with only planar bone facing surfaces.

22. The orthopaedic implant of claim 1, wherein the bone facing surface and/or bone engaging means increases a surface area of an interface between the implant and the prepared surface of the bone by up to 100% relative to an orthopaedic implant with only planar bone facing surfaces.

23. The orthopaedic implant of claim 1, wherein the bone facing surface and/or bone engaging means increases a surface area of an interface between the implant and the prepared surface of the bone by up to 1000% relative to an orthopaedic implant with only planar bone facing surfaces.

24. The orthopaedic implant of claim 1, wherein the bone facing surface of the body is configured to mate with the prepared surface of a femoral bone.

25. The orthopaedic implant of claim 1, wherein the bone facing surface of the body is configured to mate with the prepared surface of a tibial bone.

26. The orthopaedic implant of claim 1, wherein the bone facing surface of the body is configured to mate with the prepared surface of a patella bone.

27. A method for using a surgical orthopaedic implant, the method comprising the steps of:
gaining access to an implant area within a patient's body;
preparing an implant receiving surface of a bone within the implant area to receive and mate with a bone facing surface of an orthopaedic implant,
mating a body of the orthopaedic implant with the bone using a bone engaging means, said bone engaging means extending from said bone facing surface and into the prepared implant receiving surface of the bone; and
securing the orthopaedic implant onto the prepared implant receiving surface of the bone.

28. The method of claim 27, wherein preparing the implant receiving surface further comprises creating corresponding cavities on the implant receiving surface, wherein said bone engaging means extends into the corresponding cavities when said body of the orthopaedic implant is mated with the bone.

29. The method of claim 28, wherein a laser bone ablation device is used for creating the corresponding cavities on the implant receiving surface.
